(19) United States
(12) Patent Application Publication
(10) Pub. No.: US 2011/0129421 A1
(43) Pub. Date: Jun. 2, 2011

(54) MATRIX METALLOPROTEASE TARGETING NUCLEIC ACIDS

(75) Inventors: Philip Kuocherng Liu, Winchester, MA (US); Christina Huang Liu, Winchester, MA (US)

(73) Assignee: The General Hospital Corporation, Boston, MA (US)

(21) Appl. No.: 12/669,426

(22) PCT Filed: Jul. 17, 2008
(86) PCT No.: PCT/US08/70277

§ 371 Date: May 24, 2010

Related U.S. Application Data
(60) Provisional application No. 60/959,856, filed on Jul. 17, 2007.

(51) Int. Cl. A61K 49/00; A61K 31/7088; A61P 35/00; A61P 31/04; A61P 31/18; B32B 5/16

(52) U.S. Cl. 424/9.1; 514/44 R; 428/402

(57) ABSTRACT

A reporter conjugate for non-invasive detection (e.g., imaging) of matrix metalloprotease (MMP) gene expression in vivo is disclosed. The conjugate includes a targeting nucleic acid linked to a contrast agent, such as a paramagnetic label that can be used with magnetic resonance (MR) imaging. The targeting nucleic acid can be an antisense strand that hybridizes to a portion of a messenger RNA encoded by the gene whose expression is to be imaged. In some embodiments, the contrast agent is a chelated metal such as gadolinium or dysprosium. The invention also features methods to detect MMP gene expression in various tissues, including the brain.

### RELATED APPLICATION

This application claims priority to U.S. provisional Application No. 60/959,856, filed on Jul. 17, 2007, the content of which is incorporated by reference in its entirety.

### GOVERNMENT SUPPORT

The inventions described and claimed herein were made with government support under R01NS045845 and R21NS057556 (awarded by NIH). The government has certain rights in this application.

### TECHNICAL FIELD

This invention relates to imaging cellular matrix metalloprotease (MMP) nucleic acids, such as imaging the delivery, uptake, activity, and/or expression of MMP nucleic acids within cells in various tissues using, e.g., magnetic resonance (MR) imaging, and more particularly to MR imaging of gene expression in the brain.

### BACKGROUND

A number of different approaches to imaging cells have been investigated using either optical, e.g., using green fluorescent protein, bioluminescence, or near infrared fluorescence, or nuclear imaging techniques. Common limitations to these techniques are limited penetration depth (optical techniques) or spatial resolution (nuclear techniques). Recent advances in MR imaging and in particular MR microscopy have led to improved image resolution. However, compared to optical and nuclear techniques, molecular probe detection by MR is several magnitudes less sensitive. On the other hand, MR imaging offers much improved spatial resolution with anatomical precision compared to other modalities such as optical imaging, computer tomography (CT), and positron emission tomography (PET).

In all of these imaging modalities, the common goal is to deliver a suitable contrast agent or label to the relevant tissue, and more specifically into the cells. In the brain, for example, one must typically find a way to overcome the blood-brain-barrier. In addition, many known contrast agents, for example, for MR imaging, have limited permeability to cells when administered to live subjects, and as a result the limited permeability provides only a short and often unstable window for MR imaging.

MMPs are involved in brain damage following, for example, stroke (Romanic et al., Stroke, 29:1020-30, 1998) or head trauma (Shibayama et al., Acta Neurochir. Suppl., 70:220-221, 1997). MMPs have also been linked to brain injury in HIV-associated neurological diseases (Liuzzi et al., J. Neurovirol., 6:156-63, 2000). Further, MMP activity is involved in tumor metastasis and angiogenesis (John and Tuszynski, Pathol. Oncol. Res., 7:14-23, 2001).

### SUMMARY

The invention is based, in part, on the discovery that short nucleic acid sequences, e.g., phosphorothioated nucleic acid sequences, linked to one or more reporter groups to form reporter conjugates, can enter cells without the need for translocation sequences or receptors and enable the detection of MMP nucleic acids (e.g., MMP-2 or MMP-9 nucleic acids). Liposomes can be used to aid the uptake of the reporter conjugates into cells. Nucleic acids designed to target MMP nucleic acid sequences in a cell, such as an MMP messenger RNA transcribed from a target gene, can be used to image expression of cellular MMP nucleic acids non-invasively in a variety of tissues, such as tissues of the brain, liver, pancreas, heart, lung, spinal cord, prostate, breast, gastrointestinal tract, ovary, and kidney.

The reporter group can be an MR contrast agent, such as a paramagnetic label, e.g., a superparamagnetic iron oxide particle whose maximum diameter is between about 1 nm and 2000 nm, e.g., between about 2 nm and 1000 nm. In some embodiments, the maximum particle diameter is between about 10 nm and 500 nm (e.g., between about 10 nm and 200 nm, between about 20 nm and 500 nm, and between about 20 nm and 200 nm). The particle can be attached to the targeting nucleic acid through entrapment in a cross-linked dextran. In some embodiments, the paramagnetic label is a chelated metal such as Gd³⁺ or Dy³⁺.

The reporter group can also be a fluorescent label, e.g., a FITC, Texas Red, Rhodamine, or a near-infrared fluorophore (e.g., indocyanine green (ICG), Cy3 5.5, or a quantum dot). In other embodiments, the reporter group is or includes a radionuclide, e.g., ¹¹C, ¹³N, ¹⁵O, or ¹⁸F.

In one aspect, the invention features reporter conjugates for imaging cellular MMP nucleic acids (e.g., MMP-2 or MMP-9 nucleic acids) that include a single targeting nucleic acid linked to one or more reporter groups. These targeting nucleic acids and reporter groups are described in detail herein.

In another aspect, the invention features methods of imaging a cellular MMP (e.g., MMP-2 or MMP-9) nucleic acid in a tissue in vivo. The methods include obtaining a reporter conjugate including a targeting nucleic acid linked to a reporter group, wherein the targeting nucleic acid hybridizes to a target MMP nucleic acid molecule corresponding to the cellular MMP nucleic acid to be imaged; administering the reporter conjugate to the tissue in an amount sufficient to provide a detectable image; allowing sufficient time to pass to allow a sufficient amount of unbound reporter conjugate (e.g., a majority of unbound conjugate) to leave the tissue; and imaging the tissue, wherein a detectable image of the reporter group in the tissue indicates the presence of the MMP cellular nucleic acid. The target MMP nucleic acid molecule can include a messenger RNA transcribed from a target gene (e.g., an MMP-2 or MMP-9 messenger RNA), and the targeting nucleic acid can include an antisense strand that hybridizes to a portion of the messenger RNA, wherein the presence of the cellular nucleic acid indicates expression of the target MMP gene. The tissue can be, e.g., brain, heart, lung, liver, pancreas, spinal cord, prostate, breast, gastrointestinal system, ovary, or kidney tissue. The tissue can be within a patient, e.g., a human patient. The reporter group can be a superparamagnetic iron oxide particle whose maximum diameter is between about 1 nm and 2000 nm. The reporter conjugate can be administered by, e.g., intravenous injection or intra-cerebroventricular infusion.

The above-described method can be used to image tissue a human patient that has an MMP-mediated disorder, such as heart attack (cardiac arrest), stroke, head trauma (gunshot wound), multiple sclerosis, bacterial meningitis, an HIV-associated neurological disease, or a cancer. In one embodiment, the tissue is the brain and the disorder causes blood-brain barrier leakage. The method can be used to monitor or evaluating tissues, e.g., brains, of patients having an MMP-mediated disorder. In particular, the methods can be used in determining efficacy and monitoring progress of a therapeutic treatment of the patient, who has received the therapeutic treatment for the disorder. The method includes obtaining a level of the above-described reporter group in the tissue. The patient is determined to be responsive to the therapeutic treatment if the obtained level is below a pre-determined level. A pre-determined level can be obtained from a normal human without the disorder according to methods described therein.

In another aspect, the invention features reporter conjugates for imaging cellular MMP nucleic acids (e.g., MMP-2 or MMP-9 nucleic acids) that include a single targeting nucleic acid linked to one or more superparamagnetic iron oxide particles the maximum diameter of which is between about 1 nm and 1000 nm (e.g., between about 10 and 100 nm). In some embodiments, the particles include a monocrystalline iron oxide nanoparticle (MION), a superparamagnetic iron oxide nanoparticle (SPION), an ultra small superparamagnetic iron oxide particle (USPIO), or cross-linked iron oxide (CLIO) particle. The particle can be surrounded by a polymeric coating material, e.g., cross-linked dextran, carboxymethylated dextran, carboxydextran, starch, polyethylene glycol, arabinogalactan, glycosaminoglycan, organic siloxane, or sulfonated styrenedivinylbenzene, to aid in coupling of the nanoparticle to other moieties. In an example, the reporter conjugate consists essentially of a single targeting MMP nucleic acid linked to one or more paramagnetic iron oxide particles. In another example, the nucleic acid is linked to the particles via a bridge agent (e.g., biotin or avidin) that is covalently linked to the nucleic acid or the particles. The invention also features a composition containing a plurality of the above described reporter conjugates for imaging a cellular nucleic acid where each of the reporter conjugates contains only one targeting MMP nucleic acid that is linked to one or more paramagnetic iron oxide particles. The maximum diameter of the particles can be between 1 nm and 1000 nm.

In another aspect, the invention features the use of a reporter conjugate including a targeting nucleic acid linked to a reporter group, wherein the targeting nucleic acid hybridizes to a target MMP nucleic acid molecule (e.g., an MMP-2 or MMP-9 nucleic acid molecule), in the preparation of a pharmaceutical composition for imaging a cellular MMP nucleic acid in a tissue in vivo.

In another aspect, the invention features methods of imaging expression of a target MMP gene in a tissue in vivo, by obtaining a reporter conjugate including a targeting nucleic acid linked to a reporter group, wherein the targeting nucleic acid hybridizes to a target MMP nucleic acid molecule (e.g., an MMP-2 or MMP-9 nucleic acid molecule); administering the reporter conjugate to the tissue in an amount sufficient to provide a detectable image; allowing sufficient time to pass to allow a sufficient amount of unbound reporter conjugate (e.g., a majority of unbound conjugate) to leave the tissue; and imaging the tissue, wherein a detectable image of the reporter group in the tissue indicates that the target MMP gene has been expressed.

In other aspects, the invention features methods of imaging a cellular MMP nucleic acid in a tissue by obtaining a reporter conjugate including a targeting nucleic acid linked to a reporter group, wherein the targeting nucleic acid hybridizes to a target MMP nucleic acid molecule (e.g., an MMP-2 or MMP-9 nucleic acid molecule); administering the reporter conjugate to the tissue in an amount sufficient to provide a detectable image; allowing sufficient time to pass to allow a sufficient amount of unbound reporter conjugate (e.g., a majority of unbound conjugate) to leave the tissue; and imaging the tissue, wherein a detectable image of the reporter group in the tissue indicates the presence of the target MMP cellular nucleic acid.

The invention also includes methods of treating a cancer cell in a patient by obtaining a conjugate including a targeting MMP nucleic acid (e.g., an MMP-2 or MMP-9 nucleic acid) linked to an anti-cancer agent, wherein the targeting nucleic acid hybridizes to a target MMP nucleic acid molecule corresponding to the cancer cell (e.g., expressed at a greater level in the cancer cell compared to normal cells); and administering the conjugate to the patient in an amount sufficient to inhibit growth of the cancer cell. The conjugate can further include a reporter group.

The invention also includes methods of treating an MMP-mediated disorder in a patient by obtaining a conjugate including a targeting MMP nucleic acid (e.g., an MMP-2 or MMP-9 nucleic acid) linked to a therapeutic agent, e.g., a dextran-coated therapeutic agent, wherein the targeting nucleic acid hybridizes to a target nucleic acid molecule corresponding to a desired target organ or tissue, and administering the conjugate to the patient in an amount sufficient to treat the disorder. The conjugate can further include a reporter group.

In other embodiments, the invention includes methods of decreasing expression of a target MMP gene (e.g., an MMP-2 or MMP-9 gene) in a cell and, optionally, detecting or imaging a cellular nucleic acid by obtaining a reporter conjugate including a nucleic acid, e.g., a phosphorothioated nucleic acid (e.g., a phosphorothioated RNA), that decreases (e.g., is designed to decrease) expression of a target gene, and administering the conjugate to a cell in an amount sufficient to decrease expression of the target gene, and, optionally, allowing sufficient time to pass to allow a sufficient amount of unbound reporter conjugate (e.g., a majority of unbound conjugate) to leave the tissue and imaging the tissue. The nucleic acid can be, e.g., an antisense nucleic acid, a short inhibitory RNA (siRNA), a micro RNA (miRNA), or a double-stranded RNA (dsRNA).

The invention also includes methods of imaging (e.g., visualizing or locating) a cell type that expresses an MMP nucleic acid in a subject. The methods include obtaining a conjugate including a targeting nucleic acid linked to a reporter group, wherein the targeting nucleic acid hybridizes to a target MMP nucleic acid molecule (e.g., MMP-2 or MMP-9) that is expressed by the cell type to be imaged, administering the conjugate to a subject in an amount sufficient to produce a detectable image, and imaging the tissue, wherein the presence of the conjugate is indicative of the cell type. The cell type to be imaged can be, e.g., a cancer cell, a transgenic cell, or a stem cell (e.g., an embryonic stem cell).

In another embodiment, the invention includes the use of a reporter conjugate including a targeting nucleic acid linked to a reporter group, wherein the targeting nucleic acid hybridizes to a target MMP nucleic acid molecule corresponding to a cellular nucleic acid (e.g., an MMP-2 or MMP-9 nucleic acid), in the preparation of a pharmaceutical composition for imaging a cellular nucleic acid in a tissue in vivo. The reporter conjugate can further include a therapeutic agent.

In another aspect, the invention features methods of treating an MMP-mediated disorder or injury (e.g., stroke, head trauma, multiple sclerosis, bacterial meningitis, an HIV-associated neurological disease, arthritis (e.g., osteoarthritis or rheumatoid arthritis), tissue ulceration (e.g., corneal, epidermal, or gastric ulceration), abnormal wound healing, periodontal diseases, bone diseases (e.g., Paget's disease or osteoporosis) or cancer (e.g., tumor growth, metastasis, or invasion) in a patient. The methods include obtaining a targeting nucleic acid, wherein the targeting nucleic acid hybridizes to a target MMP nucleic acid (e.g., an MMP-2 or MMP-9 nucleic acid) corresponding to a target organ or tissue; and administering the targeting nucleic acid to a patient in an amount sufficient to treat the disorder. In some embodiments, the targeting nucleic acid reduces expression or activity of an MMP protein (e.g., an MMP-2 or MMP-9 protein) expressed by the target nucleic acid. The targeting nucleic acid can be, e.g., an antisense nucleic acid, a short inhibitory RNA (siRNA), a micro RNA (miRNA), or a double-stranded RNA (dsRNA). In some embodiments, the targeting nucleic acid is conjugated to a reporter group.

In some embodiments, the MMP is a gelatinase (e.g., MMP-2 or MMP-9).

A nucleic acid that hybridizes or binds “specifically” to a target nucleic acid hybridizes or binds preferentially to the target, and does not substantially bind to other molecules or compounds in a biological sample.

As used herein, “paramagnetic” means having positive magnetic susceptibility and lacking magnetic hysteresis (ferromagnetism).

As used herein, “superparamagnetic” means having positive magnetic susceptibility arid lacking magnetic hysteresis (ferromagnetism) at temperatures below the Curie or the Néel temperature of the material.

As used herein, an “MMP-mediated” disorder or injury is one that is associated, linked, connected, related, or directly or indirectly caused by expression or activity (e.g., increased or abnormal activity) of an MMP.

The new conjugates and methods allow real time imaging, such as MR imaging, and avoid the need for biopsies. The imaging is safe and can be performed as often as is needed over a period of several days.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A to 1H are a series of schematic representations of reporter conjugates, showing certain possible attachments of reporter groups, such as contrast agents or labels, that can be linked, e.g., via covalent bonds, directly or indirectly to one (FIGS. 1A-1D) or both ends (FIGS. 1E to 1H) of a double- or single-stranded nucleic acid, or with additional sites within the targeting nucleic acids. Reporter groups, e.g., contrast agents and labels that can be used include, but are not limited to, paramagnetic agents, fluorescent labels (FITC, Rhodamine, Texas Red), radioactive isotopes, individually or combinations.

FIG. 1I is a legend depicting the symbols used in FIGS. 1A to 1H.

FIG. 2A is a diagram showing an exemplary experimental protocol. Time length is not to scale.

FIG. 2B is a picture showing averaged R2* maps of mouse brains that experienced Global cerebral ischemia (GCI) for 60 min and then received SPION-mmp9 (top row) or SPION-Ran (bottom row). R2* maps were computed using a series of gradient echo of constant TR and incremental TE (TR/TE=500/3, 4, 6, 8, 10 ms), number of average=2, 117×117 μm² in plan resolution and 0.5 mm thickness. Images were co-registered before computation of pixel-wise difference/averaging.

FIG. 2C is a bar graph depicting regional SPION retention in the right (contralateral to intracerebroventricular site) hemisphere at 10 hours post bilateral carotid artery occlusion in the striatum and somatosensory cortex of animals infused with SPION-MMP9 or SPION-Ran.

FIGS. 3A-3C are two schematic representations of reporter conjugates (3A and 3B) and a legend depicting the symbols used therein (3C).

FIG. 4A is a picture showing conventional images of the brain section under investigation. Top row are pictures of diffusion weighted images (DWI) acquired one day post reperfusion using the following MRI parameters: spin echo, TR/TE=3000/27 ms, b value 1294 s/mm², number of repetition 8, 180×180 μm² in plan resolution and 1 mm thickness. Bottom row are pictures of T2 weighted spin echo images of the same animal at 5 weeks post reperfusion using the following MRI acquisition parameters: spin echo RARE, TR/TE=5000/11 ms, RARE factor=8, number of averaging=4, 117×117 μm² in plan resolution and 0.5 mm thickness.

FIGS. 4B and 4C are images of enhanced SPION retention following administration of SPION-Actin and SPION-MMP9 represented in percent R2* increase (25-150%) compared to the baseline R2* map, respectively. R2* and difference maps are constructed based on MRI acquisition protocols shown in FIG. 2B.

### DETAILED DESCRIPTION

The invention relates to new methods and compositions for detecting, e.g., imaging, the uptake/distribution and/or expression of MMP target genes in various cells and tissues, such as in the brain, non-invasively using various imaging modalities, such as MR imaging. The invention further relates to methods of reducing the expression of MMP target genes in MMP-mediated injuries and disorders, e.g., in treatment of stroke, head trauma, multiple sclerosis, bacterial meningitis, HIV-associated neurological disease, or cancer (e.g., metastatic or potentially metastatic cancer).

Shown below are nucleotide sequences of human MMP-9 transcript (SEQ ID NO:1, GenBank Accession No. NM_{—}004994) and coding region (underlined, SEQ ID NO:2):

(SEQ ID NO: 1)
AGACACCTCTGCCCTCACCATGAGCCTCTGGCAGCCCCTGGTCCTGGTG
CTCCTGGTGCTGGGCTGCTGCTTTGCTGCCCCCAGACAGCGCCAGTCCA
CCCTTGTGCTCTTCCCTGGAGACCTGAGAACCAATCTCACCGACAGGCA
GCTGGCAGAGGAATACCTGTACCGCTATGGTTACACTCGGGTGGCAGAG
ATGCGTGGAGAGTCGAAATCTCTGGGGCCTGCGCTGCTGCTTCTCCAGA
AGCAACTGTCCCTGCCCGAGACCGGTGAGCTGGATAGCGCCACGCTGAA
GGCCATGCGAACCCCACGGTGCGGGGTCCCAGACCTGGGCAGATTCCAA
ACCTTTGAGGGCGACCTCAAGTGGCACCACCACAACATCACCTATTGGA
TCCAAAACTACTCGGAAGACTTGCCGCGGGCGGTGATTGACGACGCCTT
TGCCCGCGCCTTCGCACTGTGGAGCGCGGTGACGCCGCTCACCTTCACT
CGCGTGTACAGCCGGGACGCAGACATCGTCATCCAGTTTGGTGTCGCGG
AGCACGGAGACGGGTATCCCTTCGACGGGAAGGACGGGCTCCTGGCACA
CGCCTTTCCTCCTGGCCCCGGCATTCAGGGAGACGCCCATTTCGACGAT
GACGAGTTGTGGTCCCTGGGCAAGGGCGTCGTGGTTCCAACTCGGTTTG
GAAACGCAGATGGCGCGGCCTGCCACTTCCCCTTCATCTTCGAGGGCCG
CTCCTACTCTGCCTGCACCACCGACGGTCGCTCCGACGGCTTGCCCTGG
TGCAGTACCACGGCCAACTACGACACCGACGACCGGTTTGGCTTCTGCC
CCAGCGAGAGACTCTACACCCAGGACGGCAATGCTGATGGGAAACCCTG
CCAGTTTCCATTCATCTTCCAAGGCCAATCCTACTCCGCCTGCACCACG
GACGGTCGCTCCGACGGCTACCGCTGGTGCGCCACCACCGCCAACTACG
ACCGGGACAAGCTCTTCGGCTTCTGCCCGACCCGAGCTGACTCGACGGT
GATGGGGGGCAACTCGGCGGGGGAGCTGTGCGTCTTCCCCTTCACTTTC
CTGGGTAAGGAGTACTCGACCTGTACCAGCGAGGGCCGCGGAGATGGGC
GCCTCTGGTGCGCTACCACCTCGAACTTTGACAGCGACAAGAAGTGGGG
CTTCTGCCCGGACCAAGGATACAGTTTGTTCCTCGTGGCGGCGCATGAG
TTCGGCCACGCGCTGGGCTTAGATCATTCCTCAGTGCCGGAGGCGCTCA
TGTACCCTATGTACCGCTTCACTGAGGGGCCCCCCTTGCATAAGGACGA
CGTGAATGGCATCCGGCACCTCTATGGTCCTCGCCCTGAACCTGAGCCA
CGGCCTCCAACCACCACCACACCGCAGCCCACGGCTCCCCCGACGGTCT
GCCCCACCGGACCCCCCACTGTCCACCCCTCAGAGCGCCCCACAGCTGG
CCCCACAGGTCCCCCCTCAGCTGGCCCCACAGGTCCCCCCACTGCTGGC
CCTTCTACGGCCACTACTGTGCCTTTGAGTCCGGTGGACGATGCCTGCA
ACGTGAACATCTTCGACGCCATCGCGGAGATTGGGAACCAGCTGTATTT
GTTCAAGGATGGGAAGTACTGGCGATTCTCTGAGGGCAGGGGGAGCCGG
CCGCAGGGCCCCTTCCTTATCGCCGACAAGTGGCCCGCGCTGCCCCGCA
AGCTGGACTCGGTCTTTGAGGAGCGGCTCTCCAAGAAGCTTTTCTTCTT
CTCTGGGCGCCAGGTGTGGGTGTACACAGGCGCGTCGGTGCTGGGCCCG
AGGCGTCTGGACAAGCTGGGCCTGGGAGCCGACGTGGCCCAGGTGACCG
GGGCCCTCCGGAGTGGCAGGGGGAAGATGCTGCTGTTCAGCGGGCGGCG
CCTCTGGAGGTTCGACGTGAAGGCGCAGATGGTGGATCCCCGGAGCGCC
AGCGAGGTGGACCGGATGTTCCCCGGGGTGCCTTTGGACACGCACGACG
TCTTCCAGTACCGAGAGAAAGCCTATTTCTGCCAGGACCGCTTCTACTG
GCGCGTGAGTTCCCGGAGTGAGTTGAACCAGGTGGACCAAGTGGGCTAC
GTGACCTATGACATCCTGCAGTGCCCTGAGGACTAGGGCTCCCGTCCTG
CTTTGGCAGTGCCATGTAAATCCCCACTGGGACCAACCCTGGGGAAGGA
GCCAGTTTGCCGGATACAAACTGGTATTCTGTTCTGGAGGAAAGGGAGG
AGTGGAGGTGGGCTGGGCCCTCTCTTCTCACCTTTGTTTTTTGTTGGAG
TGTTTCTAATAAACTTGGATTCTCTAACCTTT

Shown below are nucleotide sequences of mouse MMP-9 transcript (SEQ ID NO:3, GenBank Accession No. NM_{—}013599) and its coding region (underlined, SEQ ID NO:4).

(SEQ ID NO: 3)
CTCACCATGAGTCCCTGGCAGCCCCTGCTCCTGGCTCTCCTGGCTTTCG
GCTGCAGCTCTGCTGCCCCTTACCAGCGCCAGCCGACTTTTGTGGTCTT
CCCCAAAGACCTGAAAACCTCCAACCTCACGGACACCCAGCTGGCAGAG
GCATACTTGTACCGCTATGGTTACACCCGGGCCGCCCAGATGATGGGAG
AGAAGCAGTCTCTACGGCCGGCT TTGCTGATGCTTCAGAAGCAGCTCT
CCCTGCCCCAGACTGGTGAGCTGGACAGCCAGACACTAAAGGCCATTCG
AACACCACGCTGTGGTGTCCCAGACGTGGGTCGATTCCAAACCTTCAAA
GGCCTCAAGTGGGACCATCATAACATCACATACTGGATCCAAAACTACT
CTGAAGACTTGCCGCGAGACATGATCGATGACGCCTTCGCGCGCGCCTT
CGCGGTGTGGGGCGAGGTGGCACCCCTCACCTTCACCCGCGTGTACGGA
CCCGAAGCGGACATTGTCATCCAGTTTGGTGTCGCGGAGCACGGAGACG
GGTATCCCTTCGACGGCAAGGACGGCCTTCTGGCACACGCCTTTCCCCC
TGGCGCCGGCGTTCAGGGAGATGCCCATTTCGACGACGACGAGTTGTGG
TCGCTGGGCAAAGGCGTCGTGATCCCCACTTACTATGGAAACTCAAATG
GTGCCCCATGTCACTTTCCCTTCACCTTCGAGGGACGCTCCTATTCGGC
CTGCACCACAGACGGCCGCAACGACGGCACGCCTTGGTGTAGCACAACA
GCTGACTACGATAAGGACGGCAAATTTGGTTTCTGCCCTAGTGAGAGAC
TCTACACGGAGCACGGCAACGGAGAAGGCAAACCCTGTGTGTTCCCGTT
CATCTTTGAGGGCCGCTCCTACTCTGCCTGCACCACTAAAGGCCGCTCG
GATGGTTACCGCTGGTGCGCCACCACAGCCAACTATGACCAGGATAAAC
TGTATGGCTTCTGCCCTACCCGAGTGGACGCGACCGTAGTTGGGGGCAA
CTCGGCAGGAGAGCTGTGCGTCTTCCCCTTCGTCTTCCTGGGCAAGCAG
TACTCTTCCTGTACCAGCGACGGCCGCAGGGATGGGCGCCTCTGGTGTG
CGACCACATCGAACTTCGACACTGACAAGAAGTGGGGTTTCTGTCCAGA
CCAAGGGTACAGCCTGTTCCTGGTGGCAGCGCACGAGTTCGGCCATGCA
CTGGGCTTAGATCATTCCAGCGTGCCGGAAGCGCTCATGTACCCGCTGT
ATAGCTACCTCGAGGGCTTCCCTCTGAATAAAGACGACATAGACGGCAT
CCAGTATCTGTATGGTCGTGGCTCTAAGCCTGACCCAAGGCCTCCAGCC
ACCACCACAACTGAACCACAGCCGACAGCACCTCCCACTATGTGTCCCA
CTATACCTCCCACGGCCTATCCCACAGTGGGCCCCACGGTTGGCCCTAC
AGGCGCCCCCTCACCTGGCCCCACAAGCAGCCCGTCACCTGGCCCTACA
GGCGCCCCCTCACCTGGCCCTACAGCGCCCCCTACTGCGGGCTCTTCTG
AGGCCTCTACAGAGTCTTTGAGTCCGGCAGACAATCCTTGCAATGTGGA
TGTTTTTGATGCTATTGCTGAGATCCAGGGCGCTCTGCATTTCTTCAAG
GACGGTTGGTACTGGAAGTTCCTGAATCATAGAGGAAGCCCATTACAGG
GCCCCTTCCTTACTGCCCGCACGTGGCCAGCCCTGCCTGCAACGCTGGA
CTCCGCCTTTGAGGATCCGCAGACCAAGAGGGTTTTCTTCTTCTCTGGA
CGTCAAATGTGGGTGTACACAGGCAAGACCGTGCTGGGCCCCAGGAGTC
TGGATAAGTTGGGTCTAGGCCCAGAGGTAACCCACGTCAGCGGGCTTCT
CCCGCGTCGTCTCGGGAAGGCTCTGCTGTTCAGCAAGGGGCGTGTCTGG
AGATTCGACTTGAAGTCTCAGAAGGTGGATCCCCAGAGCGTCATTCGCG
TGGATAAGGAGTTCTCTGGTGTGCCCTGGAACTCACACGACATCTTCCA
GTACCAAGACAAAGCCTATTTCTGCCATGGCAAATTCTTCTGGCGTGTG
AGTTTCCAAAATGAGGTGAACAAGGTGGACCATGAGGTGAACCAGGTGG
ACGACGTGGGCTACGTGACCTACGACCTCCTGCAGTGCCCTTGAACTAG
GGCTCCTTCTTTGCTTCAACCGTGCAGTGCAAGTCTCTAGAGACCACCA
CCACCACCACCACACACAAACCCCATCCGAGGGAAAGGTGCTAGCTGGC
CAGGTACAGACTGGTGATCTCTTCTAGAGACTGGGAAGGAGTGGAGGCA
GGCAGGGCTCTCTCTGCCCACCGTCCTTTCTTGTTGGACTGTTTCTAAT
AAACACGGATCCCCAACCTTTTCCAGCTACTTTAGTCAATCAGCTTATC
TGTAGTTGCAGATGCATCCGAGCAAGAAGACAACTTTGTAGGGTGGATT
CTGACCTTTTATTTTTGTGTGGCGTCTGAGAATTGAATCAGCTGGCTTT
TGTGACAGGCACTTCACCGGCTAAACCACCTCTCCCGACTCCAGCCCTT
TTATTTATTATGTATGAGGTTATGTTCACATGCATGTATTTAACCCACA
GAATGCTTACTGTGTGTCGGGCGCGGCTCCAACCGCTGCATAAATATTA
AGGTATTCAGTTGCCCCTACTGGAAGGTATTATGTAACTATTTCTCTCT
TACATTGGAGAACACCACCGAGCTATCCACTCATCAAACATTTATTGAG
AGCATCCCTAGGGAGCCAGGCTCTCTACTGGGCGTTAGGGACAGAAATG
TTGGTTCTTCCTTCAAGGATTGCTCAGAGATTCTCCGTGTCCTGTAAAT
CTGCTGAAACCAGACCCCAGACTCCTCTCTCTCCCGAGAGTCCAACTCA
CTCACTGTGGTTGCTGGCAGCTGCAGCATGCGTATACAGCATGTGTGCT
AGAGAGGTAGAGGGGGTCTGTGCGTTATGGTTCAGGTCAGACTGTGTCC
TCCAGGTGAGATGACCCCTCAGCTGGAACTGATCCAGGAAGGATAACCA
AGTGTCTTCCTGGCAGTCTTTTTTAAATAAATGAATAAATGAATATTTA
CTT

The general methodology and reporter conjugates, as well as applications for the new reporter conjugates will be described, and the examples will show that the reporter conjugates (e.g., SPION-s-ODN), after delivery to live subjects, can be internalized by brain cells; that the retention correlates with cerebral edema following brain injury; and that targeting nucleic acids can reduce MMP expression and cerebral edema following brain injury. The examples also show that reporter constructs can be systemically administered to animals with brain injury and cross the blood-brain barrier.

### General Methodology

The new imaging methods use novel reporter conjugates to detect and/or image the uptake and distribution of MMP targeting nucleic acids, e.g., oligodeoxyribonucleotides (ODN), delivered to the brain or other tissues in live animals and humans. The conjugates include a reporter group, such as a contrast agent or a label, e.g., an MR contrast agent, e.g., iron oxide nanoparticles (e.g., SPION or MION-dextran) linked to a targeting nucleic acid (such as a single-stranded ODN) that hybridizes to a portion of a particular target nucleic acid molecule. The conjugate is delivered to the tissue containing, or thought to contain, an MMP target gene (e.g., MMP-2 or MMP-9), whose uptake, distribution, or expression is to be imaged. For example, if the reporter conjugate is to be delivered to the brain, one can use convection-enhanced delivery to the cerebral ventricles such as to the lateral ventricle (Liu et al., Ann Neurol., 36:566-76, 1994; and Cui et al., J. Neurosci., 19:1335-44, 1999) or the 4^{th} ventricles (Sandberg et al., J. Neuro-Oncology, 58:187-192, 2002). Delivery can also be intrathecal (Liu et al., Magn. Reson. Med. 51:978-87, 2004) or by any additional routes that lead directly or indirectly to brain cells. The general methodology is described in detail in WO 2006/023888.

After a sufficient amount of time for the reporter conjugate to be localized to, and internalized by, the appropriate cells within the tissue, and for a sufficient amount of unbound reporter conjugates (e.g., a majority of unbound conjugates) unbound conjugates to leave the tissue, the tissue is imaged. For example, the tissue can be imaged with a series of high-resolution T2*-weighted MR images, e.g., taken 1, 2, or 3 days after infusion of the reporter conjugate.

To use the new conjugates and methods to image gene expression, the targeting nucleic acid can be prepared as an antisense strand that is designed to hybridize to a portion of a target messenger RNA transcribed from the target gene. Thus, if a reporter conjugate including this antisense strand is detected in cells in a tissue, it provides a clear indication that that target MMP mRNA is present in the cell, and thus that the target MMP gene is being expressed.

### Reporter Conjugates

The reporter conjugates are prepared by conjugating or linking one or more MMP targeting nucleic acids to one or more reporter groups, such as magnetic particles that change the relaxivity of the cells once internalized so that they can be imaged using MR imaging. One targeting nucleic acid can have multiple (e.g., 2, 3, or more) reporter groups attached (all or some the same or different), or a set of numerous reporter conjugates can be created in which they all have the same targeting nucleic acid and 2 or more different reporter groups within the set. Alternatively, a set of reporter conjugates can be made that have different targeting nucleic acids that all target different portions of the same target gene (or that target different target genes), and each have the same or different reporter groups.

A general rationale is that each reporter conjugate must contain a sequence capable of binding to a specific target mRNA, and the conjugate must also be able to form hybrids for a period of time long enough to image transient conjugate retention or to block translation of MMP-9 protein precursor. Moreover, the does of the conjugate must be high enough to generate sufficient contrast-to-noise ratio and low enough to be cleared from a target within a reasonable span of time. In addition, the dose should not block target gene translation unless gene knockdown is the objective. Because gene transcript targeting and reporting are based on specific binding of the nucleic acid in the conjugate to its target, the conjugate must have sufficient reporting sensitivity. For example, the conjugate has sufficient reporting sensitivity when its loading capacity is one, that is, one targeting nucleic acid to one contrast agent. In the case of more than one contrast agents per nucleic acid, the sensitivity will be even higher. In contrast, four nucleic acids per contrast agent (loading capacity of 4 as seen in conventional MRI imaging) will reduce reporting sensitivity by 75%. Due to the reporting sensitivity, the conjugate described therein allows one to obtain unexpectedly specific and strong signals.

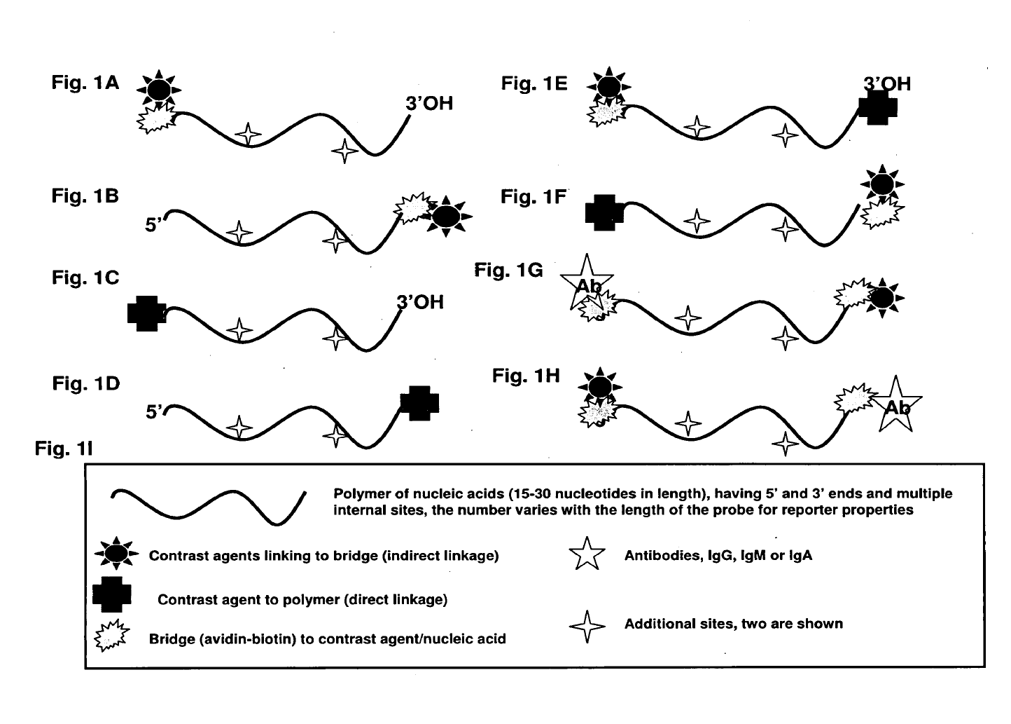

Several variations of the different types of reporter conjugates are shown in FIGS. 1A to 1I. As shown in FIGS. 1A and 1B, the conjugate includes a targeting nucleic acid of about 15 to about 30 nucleotides (also referred to herein as an oligonucleotide or ODN), one or more reporter groups, such as a contrast agent, linked to either the 5′ or 3′ ends of the ODN, either directly, e.g., by a covalent bond or via an optional linker group or “bridge” (e.g., a linkage of a desired length) between the ODN and the reporter group(s). The targeting nucleic acid typically has at least 80% sequence homology (identity) with a sequence that is complementary to a portion of the target nucleic acid molecule. For example, at least 15 nucleotides in the ODN would be complementary to a portion of the target nucleic acid, and thus will hybridize preferentially to the target nucleic acid. The targeting nucleic acid can be either single-stranded DNA or RNA, and is typically an antisense strand, and thus complementary, to a portion of the target nucleic acid. The ODN may include one or multiple internal sites that can be attached to a reporter group, e.g., labeled, for example, with a radioactive or fluorescent label. More than 50 unique reporter groups can be made in an average length (2 kilobases) of a gene transcript (mRNA). For example, 50 different reporter conjugates can be made that specifically bind to a specific target nucleic acid, e.g., to different portions of the same target. All 50 conjugates can have the same or different reporter groups, and could have different (e.g., up to 50 different) reporter groups on the 50 different conjugates. This can be used to provide signal amplification. In addition, similar numbers of reporter contrast agents can be made to the exons of a given gene.

FIGS. 1E to 1H show reporter conjugates that include two or more reporter groups, as well as an optional antibody that can be attached at either end of the molecule (FIGS. 1G and 1H). These antibodies are typically ones that bind specifically to cell-surface antigens of particular cells or cell types to direct the reporter conjugate to the appropriate cells. Once on the surface of the cell, the reporter conjugates pass through the cell membrane and into the cells, thereby delivering the reporter group into the cell. Once in the cell, the targeting nucleic acids hybridize preferentially to their specific target nucleic acid, such as an mRNA, and remain bound within the cell. Absent the targeting nucleic acid, the reporter groups are not retained within the cells.

The MMP targeting nucleic acid can be linked to the reporter group or groups by a variety of methods, including, e.g., covalent bonds, bifunctional spacers (“bridge”) such as, avidin-biotin coupling, Gd-DOPA-dextran coupling, charge coupling, or other linkers.

The reporter groups can be contrast agents such as magnetic particles, such as superparamagnetic, ferromagnetic, or paramagnetic particles. Paramagnetic metals (e.g., transition metals such as manganese, iron, chromium, and metals of the lanthanide group such as gadolinium) alter the proton spin relaxation property of the medium around them.

There is considerable latitude in choice of particle size. For example, the particle size can be between 1 nm and 2000 nm, e.g., between 2 nm and 1000 nm (e.g., 200 or 300 nm), or between 10 nm and 100 nm, as long as they can still be internalized by the cells. The magnetic particles are typically nanoparticles. Preferably, within any particular probe preparation, particle size is controlled, with variation in particle size being limited, e.g., substantially all of the particles having a diameter in the range of about 30 nm to about 50 nm. Particle size can be determined by any of several suitable techniques, e.g., gel filtration or electron microscopy. An individual particle can consist of a single metal oxide crystal or a multiplicity of crystals.

There are two types of contrast agents useful for MR imaging: T1 and T2 agents. The presence of T1 agent, such as manganese and gadolinium, reduces the longitudinal spin-lattice relaxation time (T1) and results in localized signal enhancement in T1 weighted images. On the other hand, the presence of a strong T2 agent, such as iron, will reduce the spin-spin transverse relaxation time (T2) and results in localized signal reduction in T2 weighted images. Optimal MR contrast can be achieved via proper administration of contrast agent dosage, designation of acquisition parameters such as repetition time (TR), echo spacing (TE) and RF pulse flip angles.

Specific examples of such magnetic nanoparticles include MIONs as described, e.g., in U.S. Pat. No. 5,492,814; Whitehead, U.S. Pat. No. 4,554,088; Molday, U.S. Pat. No. 4,452,773; Graman, U.S. Pat. No. 4,827,945; and Toselson et al., Bioconj. Chemistry, 10:186-191 (1999), superparamagnetic iron oxide particles (SPIOs), SPIONs, USPIOs, and CLIO particles (see, e.g., U.S. Pat. No. 5,262,176).

MIONs can consist of a central 3 nm monocrystalline magnetite-like single crystal core to which are attached an average of twelve 10 kD dextran molecules resulting in an overall size of 20 nm (e.g., as described in U.S. Pat. No. 5,492,814 and in Shen et al., “Monocrystalline iron oxide nanocompounds (MION): Physicochemical Properties,” Magnetic Resonance in Medicine, 29:599-604 (1993), to which nucleic acids can be conjugated for targeted delivery.

The dextran/Fe w/w ratio of a MION can be, e.g., about 1.6:1. R1=12.5 mM sec⁻¹, R2=45.1 mM sec⁻¹ (0.47 T, 38° C.). At room temperature relaxivity in an aqueous solution at room temperature and 0.47 Tesla can be: R1˜19/mM/sec, R2˜41/mM/sec. MIONs elute as a single narrow peak by high performance liquid chromatography with a dispersion index of 1.034; the median MION particle diameter (of about 21 nm as measured by laser light scattering) corresponds in size to a protein with a mass of 775 kD and contains an average of 2064 iron molecules.

The physicochemical and biological properties of the magnetic particles can be improved by crosslinking the dextran coating of magnetic nanoparticles to form CLIOs to increase blood half-life and stability of the reporter complex. The cross-linked dextran coating cages the iron oxide crystal, minimizing opsonization. Furthermore, this technology allows for slightly larger iron cores during initial synthesis, which improves the R2 relaxivity. CLIOs can be synthesized by crosslinking the dextran coating of generic iron oxide particles (e.g., as described in U.S. Pat. No. 4,492,814) with epibromohydrin to yield CLIOs as described an U.S. Pat. No. 5,262,176.

The magnetic particles can have a relaxivity on the order of 35 to 40 mM/sec, but this characteristic depends upon the sensitivity and the field strength of the MR imaging device. The relaxivities of the different reporter conjugates can be calculated as the slopes of the curves of 1/T1 and 1/T2 vs. iron concentration; T1 and T2 relaxation times are determined under the same field strength, as the results of linear fitting of signal intensities from serial acquisition: (1) inversion-recovery MR scans of incremental inversion time for T1 and (2) SE scans of a fixed TR and incremental TE. Stability of the conjugates can be tested by treating them under different storage conditions (4° C., 21° C., and 37° C. for different periods of time) and performing HPLC analysis of aliquots as well as binding studies.

In some embodiments, the paramagnetic label is a metal chelate. Suitable chelating moieties include macrocyclic chelators such as 1,4,7,10-tetrazazcyclo-dodecane-N,N′,N″,N′″-tetraacetic acid (DOTA). For use in vivo, e.g., as MR contrast agents in a human patient, gadolinium (Gd³⁺), dysprosium (Dy³⁺), and europium are suitable. Manganese can also be used for imaging tissues other than in the brain. In other embodiments, CEST (Chemical Exchange Saturation Transfer) can be used. The CEST method uses endogenous compounds such as primary amines as reporter groups that can be linked to the ODN.

Other suitable reporter groups are labels such as near infrared fluorophores, e.g., indocyanine green (ICG) and Cy5.5 and quantum dots, which can be linked to the targeting nucleic acid and used in optical imaging techniques, such as diffuse optical tomography (DOT) (see, e.g., Ntziachristos et al., Proc. Natl. Acad. Sci. USA, 97:2767-2773, 2000). Other fluorescent labels, such as FITCs, Texas Red, and Rhodamine can also be linked to the targeting nucleic acid. Radionuclides, such as ¹¹C, ¹³N, ¹⁵O or ¹⁸F, can be synthesized into the targeting nucleic acids to form the reporter conjugates. In addition, various known radiopharmaceuticals such as radiolabeled tamoxifen (used, e.g., for breast cancer chemotherapy) and radiolabeled antibodies can be used. For example, they can be coated with dextran for attachment to the targeting nucleic acids as described herein. These radio-conjugates have application in positron emission tomography (PET). Radioisotopes, such as ³²P, ³³P, ³⁵S (short half-life isotopes) (Liu et al. (1994) Ann. Neurol., 36:566-576), radioactive iodine, and barium can also be integrated into or linked to the targeting nucleic acid to form conjugates that can be imaged using X-ray technology.

Note that two or more reporter groups, of the same or different kinds, can be linked to a single targeting nucleic acid.

The targeting nucleic acids are typically single-stranded, antisense oligonucleotides of 12, 15, 18, 20, 23, 25, 26, 27, and up to about 30 nucleotides in length. They are designed to hybridize to the target gene (if present in sufficient numbers in a cell), or to hybridize to a messenger RNA transcribed from the gene whose expression is to be imaged. They can be protected against degradation, e.g., by using phosphorothioate, which can be included during synthesis. In addition, by keeping the length to about 30 or fewer nucleotides, the non-specific nuclease/protease response that could destroy cellular mRNA and induce a cytotoxic reaction can be avoided.

The reporter group and the targeting nucleic acid are then linked to produce the reporter conjugate using any of several known methods. For example, if the contrast agent is a MION, this molecule can be linked to a nucleic acid by phosphorothioating the oligonucleotide and labeling it with biotin at the 5′ end. The dextran coated MION can be activated and conjugated to the biotin-labeled oligonucleotide using avidin based linkers, such as NeutrAvidin® (Pierce Chem.) or other Avidin derivatives such as StratAvidin.

In addition, liposomes, lipofectin, and lipofectamine can be used to help get the entire conjugate into a cell.

Various imaging modalities and corresponding reporter groups are reviewed and described in Min et al., Gene Therapy, 11:115-125 (2004), which is incorporated herein by reference in its entirety, including the references it cites.

### MMP Targeting Nucleic Acids

MMP targeting nucleic acids include isolated nucleic acid fragments sufficient for use as hybridization probes to identify the nucleic acid molecules encoding MMPs (e.g., MMP-2 or MMP-9) in a sample or in a cell, as well as nucleotide fragments for use as PCR primers for the amplification or mutation of the nucleic acid molecules described herein. As used herein, the term “nucleic acid molecule” is intended to include DNA molecules (e.g., cDNA or genomic DNA) and RNA molecules (e.g., mRNA) and analogs of the DNA or RNA (e.g., phosphorothioated analogs) generated using nucleotide analogs. The nucleic acid can be single-stranded or double-stranded, but preferably is double-stranded DNA.

A targeting nucleic acid molecule, e.g., a nucleic acid molecule having the nucleotide sequence of an MMP transcript (e.g., MMP-2 or MMP-9) or a portion thereof, can be isolated using standard molecular biology techniques and the sequence information provided herein. Furthermore, oligonucleotides corresponding to an MMP transcript (e.g., MMP-2 or MMP-9) can be prepared by standard synthetic techniques, e.g., using an automated DNA synthesizer. A targeting nucleic acid can be constructed using chemical synthesis and enzymatic ligation reactions using procedures known in the art. Targeting nucleic acids can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl)uracil, (acp3)w, and 2,6-diaminopurine.

In one embodiment, a targeting nucleic acid molecule comprises a nucleic acid molecule that is a complement of the nucleotide sequence of an MMP transcript (e.g., MMP-2 or MMP-9) or a portion of any of these nucleotide sequences.

The targeting nucleic acids, moreover, can comprise only a portion of the nucleic acid sequence of an MMP transcript (e.g., MMP-2 or MMP-9) or the complement thereof. The targeting nucleic acid typically comprises a region of nucleotide sequence that hybridizes under stringent conditions to at least about 7, 8, 9, 10, 11, 12, 13, 14, 15, 18, 21, 24, 27, 30, 35, 40, 45, or 50 consecutive nucleotides of an MMP transcript (e.g., MMP-2 or MMP-9) or the complement thereof.

Nucleic acid molecules corresponding to natural allelic variants and homologues of MMP transcripts (e.g., MMP-2 or MMP-9) can be isolated based on their homology to the MMP genes (e.g., MMP-2 or MMP-9) using the sequences disclosed herein, or a portion thereof, as a hybridization probe according to standard hybridization techniques under stringent hybridization conditions. Nucleic acid molecules corresponding to natural allelic variants and homologues of the marker genes can further be isolated by mapping to the same chromosome or locus as the marker genes or genes encoding the marker proteins.

In another embodiment, an isolated targeting nucleic acid molecule is at least 7, 8, 9, 10, 11, 12, 13, 14, 15, 18, 21, 24, 27, 30, 35, 40, 45, or 50 nucleotides in length and hybridizes under stringent conditions to a nucleic acid molecule corresponding to an MMP transcript (e.g., MMP-2 or MMP-9). As used herein, the term “hybridizes under stringent conditions” is intended to describe conditions for hybridization and washing in 6×SSC at about 45° C., followed by one or more washes in 0.2×SSC, 0.1% SDS at 65° C. Guidance on designing nucleic acids that hybridize to a target under specific conditions (e.g., intracellular conditions) can be found, e.g., in Ausubel et al., eds. Current Protocols in Molecular Biology, John Wiley & Sons, N.Y. (1989).

Another aspect relates to isolated nucleic acid molecules that are antisense to an MMP transcript (e.g., MMP-2 or MMP-9). An “antisense” nucleic acid comprises a nucleotide sequence that is complementary to a “sense” nucleic acid encoding a protein, e.g., complementary to the coding strand of a double-stranded cDNA molecule or complementary to an mRNA sequence. Accordingly, an antisense nucleic acid forms hydrogen bonds to a sense nucleic acid. The antisense nucleic acid can be complementary to an entire coding strand of the MMP transcript (e.g., MMP-2 or MMP-9), or to only a portion thereof. In one embodiment, an antisense nucleic acid molecule is antisense to a “coding region” of the coding strand of a nucleotide sequence described herein. The term “coding region” includes the region of the nucleotide sequence comprising codons that are translated into amino acid. In another embodiment, the antisense nucleic acid molecule is antisense to a “noncoding region” of the coding strand of a nucleotide sequence described herein. The term “noncoding region” includes 5′ and 3′ sequences that flank the coding region that are not translated into amino acids (i.e., also referred to as 5′ and 3′ untranslated regions).

Antisense nucleic acids can be designed according to the rules of Watson and Crick base pairing. The antisense nucleic acid molecule can be complementary to the entire coding region of an mRNA corresponding to a gene described herein, but can also be an oligonucleotide that is antisense to only a portion of the coding or noncoding region. An antisense oligonucleotide can be, for example, about 5, 7, 10, 15, 20, 25, 30, 35, 40, 45 or 50 nucleotides in length. An antisense nucleic acid can be constructed using chemical synthesis. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules described herein are typically administered to a subject or generated in situ such that they hybridize with or bind to cellular mRNA and/or genomic DNA encoding an MMP protein (e.g., MMP-2 or MMP-9), thereby inhibiting expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix.

In yet another embodiment, the antisense nucleic acid molecules described herein are I-anomeric nucleic acid molecules. An I-anomeric nucleic acid molecule forms specific double-stranded hybrids with complementary RNA in which, contrary to the usual σ-units, the strands run parallel to each other (Gaultier et al. (1987) Nucleic Acids. Res. 15:6625-6641). The antisense nucleic acid molecules can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

In still another embodiment, an antisense nucleic acid can be a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach (1988) Nature 334:585-591)) can be used to catalytically cleave mRNA transcripts, to thereby inhibit translation of this mRNA. A ribozyme having specificity for a marker protein-encoding nucleic acid can be designed based upon the nucleotide sequence of an MMP transcript (e.g., MMP-2 or MMP-9).

Alternatively, expression of MMP genes (e.g., MMP-2 or MMP-9) can be inhibited by targeting nucleotide sequences complementary to the regulatory region of these genes (e.g., the promoter and/or enhancers) to form triple helical structures that prevent transcription of the gene in target cells. See generally, Helene (1991) Anticancer Drug Des., 6:569-84; Helene et al. (1992) Ann. N.Y. Acad. Sci. 660:27-36; and Maher (1992) Bioassays, 14:807-15.

The term siRNA refers to small inhibitory RNA duplexes that induce the RNA interference (RNAi) pathway. These molecules can vary in length (generally between 18-30 nucleotides) and contain varying degrees of complementarity to their target mRNA in the anti sense strand. Some, but not all, siRNA have unpaired overhanging bases on the 5′ or 3′ end of the sense strand and/or the antisense strand. The term siRNA includes duplexes of two separate strands, as well as single strands that can form hairpin structures comprising a duplex region. See, e.g., Elbashir et al. (2001) Nature, 411: 494-8; Birmingham et al. (2006) Nat. Methods, 3:199-204; Chakraborty (2007) Curr. Drug Targets, 8:469-82; and Patzel (2007) Drug Discov. Today, 12:139-48.

### Methods of Administration

For administration, e.g., to an experimental rodent or human patient, a reporter conjugate can be diluted in a physiologically acceptable fluid such as buffered saline, dextrose or mannitol. Preferably, the solution is isotonic. Alternatively, the conjugate can be lyophilized and reconstituted with a physiological fluid before injection. The conjugate can be administered parenterally, e.g., by intravenous (IV) injection, subcutaneous injection, or intra-muscular administration, depending on the tissue to be imaged. For imaging the brain, a useful route of administration is the intracerebroventricular (ICV) route. When administered intravenously (IV) or intraperitoneally (i.p.), the conjugate can be administered at various rates, e.g., as rapid bolus administration or slow infusion.

When administered by IV injection and superparamagnetic iron particles are used as the paramagnetic label, useful dosages are between about 0.1 and 10.0 mg of iron per kg, e.g., between 0.2 and 5 mg/kg for a 1.5 Tesla medical scanner. As is known in this art, there is a field dependence component in determining the contrast dosage. Doses of iron higher than 10 mg/kg should be avoided because of the inability of iron to be excreted. These types of contrast agents can be used at a dosage of 0.001 to 0.1 mg/kg body weight for ICV administration in the rodents.

When administered by IV injection and chelated gadolinium is used as the paramagnetic label, the dose will be between 10 micromoles and 1000 micromoles gadolinium/kg, e.g., between 50 and 100 micromoles gadolinium/kg. Doses above 1000 micromoles/kg produce hyperosmotic solutions for injection.

The new reporter conjugates will shorten the relaxation times of tissues (T1 and/or T2) and produce brightening or darkening (contrast) of MR images of cells, depending on the tissue concentration and the pulse sequence used. In general, with highly T2 weighted pulse sequences and when iron oxides are used, darkening will result. With T1 weighted pulse sequences and when gadolinium chelates are used, brightening will result. Contrast enhancement will result from the selective uptake of the conjugate in cells that contain the target gene.

If delivered systemically, paramagnetic metal chelate-type conjugates will show renal elimination with uptake by the liver and spleen, and to a less degree by other tissues.

Superparamagnetic iron oxide crystal-type conjugates are too large for elimination by glomerular filtration. Thus, most of the administered conjugate will be removed from the blood by the liver and spleen. Superparamagnetic iron oxides are biodegradable, so the iron eventually will be incorporated into normal body iron stores.

Various reporter groups for medical imaging are routinely administered to patients intravenously, but can also be delivered by intra-peritoneal, intravenous, or intra-arterial injection. All of these methods can deliver the new reporter conjugates throughout the body except to the brain due to the existence of the blood brain barrier (BBB). To bypass the BBB, one can either use ICV, or one can employ intrathecal injection into the Cisterna Magna, or intra-arterial injection into the ascending aorta, followed by the transient breakage of BBB (e.g., via mannitol infusion). In certain situations, the BBB may be already breached because of a specific disorder, such as brain injury or certain cancers.

### Methods of Imaging

MR imaging can be performed in live animals or humans using standard MR imaging equipment, e.g., clinical, wide bore, or research oriented small-bore MR imaging equipment, of various field strengths. Imaging protocols typically consist of T₁, T₂, and T₂* weighted image acquisition, T1 weighted spin echo (SE 300/12), T2 weighted SE (SE 5000/variable TE) and gradient echo (GE 500/variable TE or 500/constant TE/variable flip angles) sequences of a chosen slice orientation at different time points before and after administration of the reporter conjugate.

To determine the in vivo distribution of a particular reporter conjugate, biodistribution studies and nuclear imaging can be carried out using excised tumors of animals that have received a single dose of labeled reporter complex, e.g., MION-s-ODN. The same assay can be used to analyze the biodistribution of other new reporter conjugates.

To determine whether expression of a specific target gene, e.g., a therapeutic transgene, can be detected with a particular reporter conjugate, animals receive an infusion of the conjugate. After injection, differences in R2* maps (inverse of T2* maps) are determined after a pre-defined period of time. If significant, the reporter conjugate can be used in clinical imaging of that specific transgene. Biodistribution studies can be used to show a higher concentration of the reporter conjugate in cells expressing the target gene compared to matched cells that do not express (or over-express) the target gene in the same animal.

This image evaluation technique can also applied to other imaging modalities such as PET, X-ray, and DOT, in which radionuclides, radioisotopes, and/or fluorescent conjugates are detected. Such other imaging modalities, and their corresponding reporter groups, are described in Min et al. (Gene Therapy, 11:115-125 (2004)).

### Applications

The new methods and compositions have numerous practical applications. The availability of reporter conjugates to detect, e.g., image, cellular nucleic acids, e.g., to image gene expression, is important for monitoring gene therapy where exogenous genes are introduced to ameliorate a genetic defect or to add an additional gene function to cells.

The new methods can also be used to image endogenous gene expression during development and/or pathogenesis of an MMP-mediated disease or injury (e.g., stroke, head trauma, multiple sclerosis, bacterial meningitis, an HIV-associated neurological disease, or a cancer).

The new methods can also be used for detecting, e.g., imaging MMP (e.g., MMP-2 or MMP-9) gene expression in deep organs using MR imaging, and for imaging tumors that over-express MMP target genes compared to normal cells. Such tumors are likely to have elevated angiogenic and/or metastatic potential compared to tumors with lower MMP expression.

Moreover, imaging of gene expression by high-resolution MR imaging will have a major impact in the treatment of CNS disease such as brain tumors or neurodegenerative diseases such as Alzheimer's. First, the new reporter conjugates can be used for in vivo monitoring of MMP (e.g., MMP-2 or MMP-9) gene expression. This will have direct applications in determining efficacy and persistence of therapy by non-invasive imaging and imaging MMP gene expression over time in the same subject. For example, the new reporter conjugates can be used before, after, or during a course of therapy or treatment for an MMP-mediated disease or injury (e.g., stroke, head trauma, multiple sclerosis, bacterial meningitis, an HIV-associated neurological disease, or a cancer), e.g., to monitor the progress of treatment.

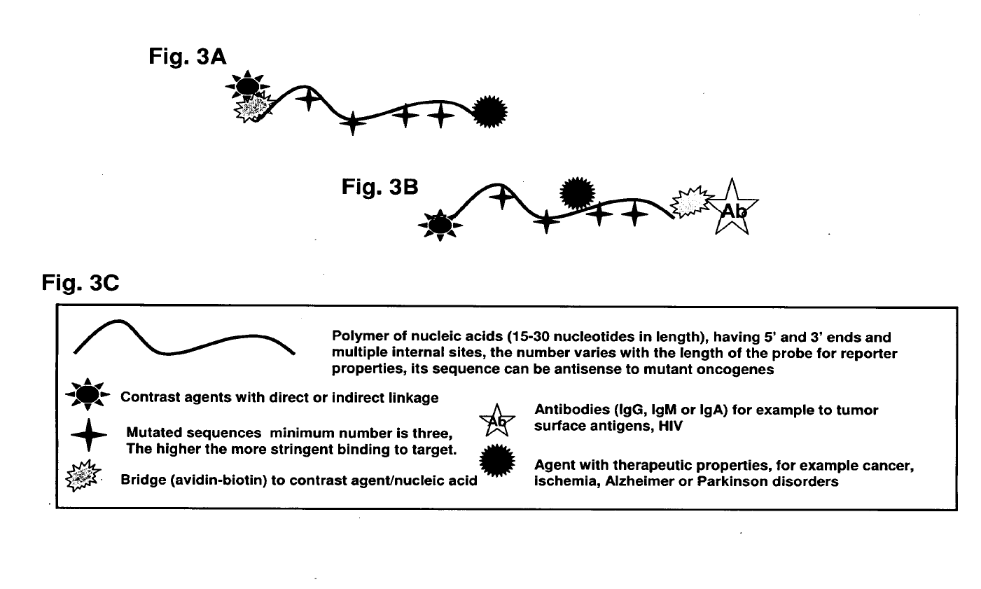

We note that a change of as few as 3 nucleotides in an ODN of 26 nucleotides in length will significantly reduce binding of the ODN to the wild-type target mRNA (Liu et al., Ann. Neurology, 36:566-576, 1994) in the brain of live animals. Therefore, one can alter a given ODN sequence so that its binding to a mutant mRNA transcribed from a mutant MMP gene will remain at 100% homology, while binding to a wild-type mRNA transcribed from a normal MMP gene is reduced. This type of targeting nucleic acid can be used to detect mutant MMP target genes. Moreover, this reporter conjugate can also be used in cancer therapy. The mutant ODN can be synthesized to be complementary to a mutated oncogene, and can be designed to carry one or more anti-cancer agents, such as radiopharmaceuticals or radioisotopes that can inhibit or kill the cancer cell (see FIGS. 3A-C). As shown in FIGS. 3A and B, the conjugates include a targeting nucleic acid of 15 to 30 nucleotides ODN; one or more reporter groups, such as a contrast agent, linked to either the 5′ or 3′ ends of the ODN, either directly, e.g., by a covalent bond or via an optional linker group or “bridge” (e.g., a linkage of a desired length) between the ODN and the reporter group(s); one or more agents with cancer therapeutic properties; optionally, one or more antibodies to tumor surface antigens; and, optionally, one or more (e.g., three or more) point mutations in the sequence.

Furthermore, the ability of the reporter conjugate to discriminate between the mutant copy and wild-type copy of a target mRNA transcribed from an oncogene can be used to enable the reporter conjugate to preferentially bind to the mutant mRNA, thereby inhibiting translation of the mutant mRNA into a gene product, and thus inhibit expression of the mutant oncogene.

The new methods can also be used for treatment of an MMP-mediated disorder or injury (e.g., stroke, head trauma, multiple sclerosis, bacterial meningitis, an HIV-associated neurological disease, or a cancer) in a patient. These disorders and injuries are often characterized by an increase in MMP activity or expression. A patient having or at risk for an MMP-mediated disorder or injury is administered a nucleic acid that reduces expression or activity of an MMP protein (e.g., an MMP-2 or -9 protein), e.g., to decrease tissue damage due to MMP activity. The nucleic acid can be administered in combination with (e.g., before, after, or at the same time as) one or more standard treatments for the disorder or injury.

In other embodiments, the new reporter conjugates can be used to detect the expression of an MMP transgene in a subject. The new reporter conjugates can be used to localize expression of a transgene in a subject. The expression of a transgene that is expressed conditionally (e.g., from a conditional promoter) or tissue specifically (e.g., from a tissue-specific promoter) can be imaged using the new reporter conjugates.

The new reporter conjugates made with either DNA or RNA as the targeting nucleic acid can also be used to deliver any reporter molecule to any specific cellular nucleic acid, such as a gene, in a collection of cellular nucleic acids, such as a gene bank.

The specific examples below are to be construed as merely illustrative, and not limitative of the remainder of the disclosure in any way whatsoever. Without further elaboration, it is believed that one skilled in the art can, based on the description herein, utilize the present invention to its fullest extent.

### Example 1

### Preparation of SPION-s-ODN

Phosphorothioated oligodeoxynucleotides (s-ODN) were labeled with biotin as the targeting nucleic acid portions of reporter conjugates. Antisense s-ODN were synthesized to bind to cfos (5′-catcatggtcgtggtttgggcaaacc-3′; SEQ ID NO:5), actin (5′-gagggagagc-atagccctcgtagatg-3′; SEQ ID NO:6), and MMP-9 (5′-tacatgagcgcttccggcac-3′; SEQ ID NO:7) mouse mRNA. An s-ODN with a random sequence having no known cellular target was also synthesized (5′-gggatcgttcagagtcta-3′; SEQ ID NO:8; Zhang et al., J. Nucl. Med., 42:1660-9, 2001). The mouse MMP-9 sequence corresponds to the human antisense sequence 5′-tacatgagcgcctccggcac-3′ (SEQ ID NO:9). In some experiments, s-ODN were synthesized with biotin at the 5′ end.

SPION were prepared for these studies as described previously (Lind et al., J. Drug Target., 10:221-30, 2002). Freshly synthesized SPION was functionalized using cyanogen bromide (Marshall and Rabinowitz, J. Biol. Chem., 251:1081-1087, 1976), and linked to NeutrAvidin (NA) in the presence of 1 M sodium cyanoborohydride (both from Pierce Biotechnology, Rockford, Ill.). The resulting covalently linked product, SPION-NA, was filtered and dialyzed against a 20× volume of sodium citrate buffer solution (25 mM, pH 8.0), using a Centricon Plus-100 filter (100 KD cut-off, Millipore Corp., Bedford, Mass.). Activated SPION (SPION-NA) was stored in an amber-colored bottle at 4° C., at a concentration of 4 mg iron per ml sodium citrate buffer. Stored in this way, activated SPION has a shelf life of up to six months. The resulting SPION-NA should optimally have one biotin binding site available to biotinylated s-ODN so that a reporter conjugate had a loading capacity of one, that is, one SPION per each s-ODN

Iron concentrations in SPION samples were determined by optical absorbance at 410 nm after treatment with hydrogen peroxide (0.03%) and 6N hydrogen chloride (de Marco et al., Radiology, 208:65-71, 1998). All sODN were purified using polyacrylamide gel electrophoresis. To directly observe the sODN, we also synthesized sODN with FITC on the 5′ terminus and biotin on the 3′ terminus (FITC-sODN-biotin). SPION-NA (250 nmol Fe) was incubated with the biotinylated sODN (FITC-sODN-biotin, 1 nmol) at room temperature for 30 minutes and the mixture was filter-dialyzed with 3 washes of sodium citrate buffer (25 mM, pH 8) in a centrifugal filter device (Microcon© YM-50, Millipore). They were then re-suspended in 36 μl sodium citrate buffer, followed by the addition of 4 μl of lipofectin (1 mg/ml, Invitrogen Life Technologies), which has been shown to facilitate sODN uptake (Cui et al., J. Neurosci., 19:1335-1344, 1999). After conjugation, the probes were stored at 4° C.

To demonstrate physical linkage, FITC-sODN-biotin (500 pmol) was incubated with SPION-NA (54 pmol) at room temperature for one hour, and the sample was resolved using gel electrophoresis. Binding of sODN to SPION retarded the migration of sODN in agarose gel (gel shift assay). Additionally, gel slices from a sample of SPION-cfos contained significant T₂ susceptibility agents, whereas unbound FITC-sODN did not. These results indicate linkage between SPION and FITC-sODN.

### Example 2

### General Animal Methods

Global cerebral ischemia (GCI) was induced in mice by transient bilateral carotid artery occlusion (BCAO). It was known that GCI induced in mice by BCAO leads to oxidative stress (Huang et al. 2000. Faseb J 14:407-417.2), a condition that in turn causes metabolic disturbance that manifests as decreased intracellular pH and adenosine triphosphate concentration and increased levels of extracellular glutamate, intracellular calcium, and reactive species of oxygen and nitrogen. Unlike that in the more common stroke model, brain damage in the GCI model seldom produces necrosis and has no ischemic core or penumbra in the damage site.

All procedures and animal care practices adhered strictly to Association for Assessment and Accreditation of Laboratory Animal Care (AAALAC), Society for Neuroscience, and institutional guidelines for experimental animal health, safety, and comfort. After anesthetizing male C57Black6 mice (25±2 g, Taconic Farm, Germantown, N.Y.) with a mixture of ketamine (80 mg/kg, i.p.) and xylazine (12 mg/kg, i.p.), a midline ventral incision was made in the neck. Both common carotid arteries were isolated, freed of nerve fibers, and occluded for 60 minutes using nontraumatic aneurysm clips (Fine Science Tools, Inc). The occlusion was released for reperfusion as described previously (Liu et al., J. Neurosci., 16:6795-6806, 1996). Complete occlusion (no blood flow) or blood flow restoration was directly observed under a surgical microscope (Zeiss OPMI-6, Carl Zeiss Microimaging, Inc., Thornwood, N.Y.). Sham-operated animals underwent the same surgical procedure except for actual execution of artery occlusion. Body temperature was monitored and maintained at 37±1° C. throughout surgery and the immediate postoperative period, until the animals recovered fully from anesthesia. The mortality rate was similar to that reported previously (Liu et al., J. Neurosci., 16:6795-6806, 1996; Barone et al., J. Cereb. Blood Flow Metab., 13:683-692, 1993).

Pre-contrast MR images were obtained for a group of at least 4 animals, as estimated by retrospective power calculation (StatPages.org/postpowr.html). Forty-eight picomoles (pmol) of SPION-NA were mixed with an equal molar ratio of biotinylated sODN or dNTP at room temperature for one hour and diluted to 1.5 pmol SPION (or 0.5 μg Fe) per microliter in sodium citrate buffer (25 mM, pH 8). Various contrast agents were delivered in 2 μl sodium citrate buffer to the lateral ventricle of anesthetized mice at a constant dose at 0.2 μl per minute (LR: −1.0; AP: −0.4; DV: −3 mm Bregma) using a Hamilton syringe (model 700 with Gauge 26S, OD=0.47 mm) and an automatic micropump on a stereotaxic device (Stoelting). All animals were scanned within 30 minutes of infusion to verify ICV delivery and to eliminate from further study any animals that received intracerebral injection. Eliminating these animals reduced the possibility of a blooming effect caused by trapped SPION in the infusion track (Bulte et al., Proc. Natl. Acad. Sci. USA, 96:15256-61, 1999). The initial scanning also verified mixing of contrast agent with cerebrospinal fluid (CSF) in the ventricular space for distribution in the brain.

Brain edema developed in various regions of the brain after BCAO of 60 minutes, and appeared consistently in the striatum and septal nucleus. Edematous volume in the striatum is biphasic in nature (N=44). At 1.5 hours (reperfusion), edema volumes were 16±2.5 mm³; they decreased to 2.4±0.9 mm³ (ADC=4.4×10⁻⁴) at 8 hours, increased significantly to 9.1±0.89 mm³ (ADC=4.3×10⁻⁴) at 11 hours and remained the same (8±1.4 mm³) for a few days thereafter. Volumes gradually fell to 3.1±0.6 mm³ in about 5 days. It was found that blood-brain barrier (BBB) leakage appeared at the first half hour of reperfusion by Evans blue extravasation (1% 0.1 ml, IV, 3 hours, N=3), and by MR T1 contrast agent in live subjects. These results are similar to BBB disturbance seen in a rat model of cardiac arrest. Diffusion weighted images (DWI) were not evaluated in the cortex because the surface coil may cause T2 shine-through artifact in that region. However, it was found that apparent diffusion coefficients (ADC) were reduced to below threshold levels. Other regions, such as the thalamus and hypothalamus, were also found to be vulnerable. The mortality rate for C57Black6 mice treated with 60-minute BCAO was 14% (N=22); mortality was not elevated in those animals that received SPION-sODN at the 28-μg per kg dose after BCAO (N=17).

In vivo image acquisition was performed with 9.4 Tesla MRI (Bruker Avance system, Bruker Biospin MRI, Inc., Billerica, Mass.) at different post-infusion time points. Animals were anesthetized with 2% isoflurane in pure O₂ (800 ml/min flow rate), and blood oxygenation levels were monitored by pulse oximetry. A one-inch surface coil was used for excitation and signal detection. Serial gradient echo (GE) images were used with a constant repetition time (TR=500 ms) and incremental echo spacing (TE=3, 4, 6 ms) to construct R2* maps (R2*=1/T2*) for a 500-μm thick slice at a resolution of 120 μm in the image plane.

DWI were acquired and ADC were computed with the following sequence parameters: TR/TE=3000/27 ms, b=154, 1294 s/mm², 180×180 μm² in-plane resolution and 1 mm slice thickness for assessment of tissue injury. Images at b=1294 s/mm² (DWI) and maps of ADC were calculated with the equation M=Mo×exp(−b ADC). Postmortem image acquisition can be performed using a 14 Tesla MRI scanner (Bruker Avance system, Bruker Biospin MRI, Inc., Bellerica, Mass.). The brains were immersed in 1-cm NMR tubes in perfluoro compound solution FC-40 to eliminate background proton signals. A one-cm volume coil was used with a fast low-angle shot (FLASH) gradient echo sequence to obtain 3-dimensional high-resolution MR images (TR/TE=50/18 ms, 40×40×40 μm³, flip angle 20 degrees).

The in vivo MR images were co-registered, and the mean R2* maps of sham-operated and BCAO-treated animals were computed using in-house software (Martinos Center for Biomedical Imaging at MGH). We chose to use the R2* values, defined as the inverse of T2* values which positively correlate to localized iron concentration.

### Example 3

### Retention of SPION-mmp9 Correlates with Cerebral Edema

To demonstrate probe applicability for neurological research applications in live animals, an antisense sODN of matrix metalloprotease-9 mRNA (sODN-mmp-9) conjugated to SPION-NA was developed. Elevation of MMP-9 is observed in brain damage following stroke. SEQ ID NO:7 has been shown to hybridize to mmp9 mRNA (Zechel et al., J. Neurosci. Res., 69:662-8, 2002).

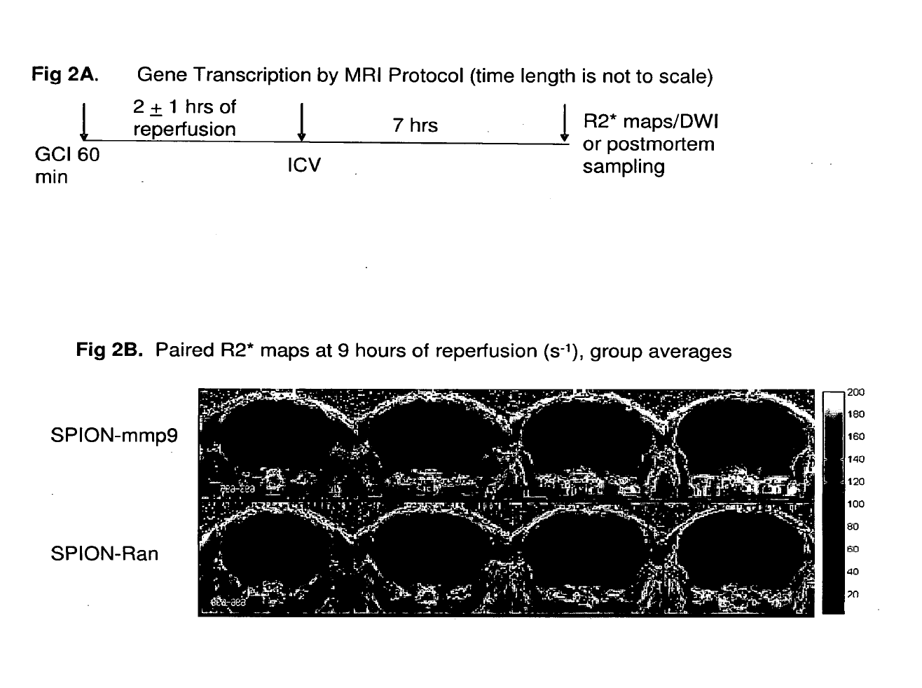

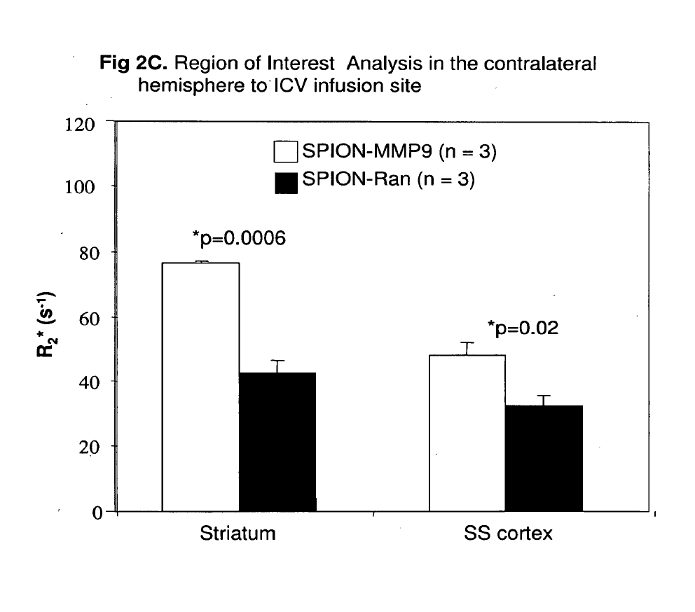

Following BCAO, 120 pmol/kg SPION-mmp9 or SPION-Ran (N=3, each) was infused at two hours of reperfusion; R2* maps were obtained 9 hours after reperfusion, before the second peak time for DWI appearance (FIG. 2A). Any lethality was attributed to increasing intracranial pressure due to infusion. No significant difference in R2* values was observed between the left and right hemispheres (cortex, p=0.09; striatum p=0.18 for animals received SPION-mmp9, p>0.25 for those that received SPION-Ran). The group averaged retention profiles of SPION-mmp9 (presented as R2* maps, FIG. 2B, top row) was pronouncedly more localized in the striatum, where DWI hyperintensity and BBB leakage were observed, compared to group averaged retention profiles of SPION-Ran (FIG. 2B, bottom row) The retention characteristics of SPION-mmp9 was different from those of both c-fos, which is expressed in the hippocampus and cortex, and actin, which is constitutively expressed, and shows no elevation in subtraction maps.

To quantitatively analyze SPION retention, R2* values were obtained of four 1-mm MR slices from each animal. The overall BCAO-induced SPION retention in the striatum and somatosensory cortex (SSC) of all three BCAO animals that received SPION-mmp9 were significantly higher than in BCAO animals that received SPION-Ran (FIG. 2C). Within the group of BCAO animals that received SPION-mmp9, SPION-mmp9 retention was higher in the striatum than in the cortex of the same group which suggested that mmp-9 mRNA expression is heavily involved in the development of striatal cerebral edema in a cardiac arrest model of C57Black6 mice. This example demonstrates that MMP expression can be detected in vivo using mmp9 mRNA targeting SPION probes and positively correlate with cerebral edema in a model of stroke.

### Example 4

### Reduction of MMP-9 Activity Reduces Tissue Damage in Cerebral Ischemia

To demonstrate that reduction of MMP-9 activity could reduce tissue damage, gene knockdowns were performed by infusion of antisense sODN-mmp9 or non-targeting sODN-Ran (Cui et al., J. Neurosci., 19:1335-1344, 1999). Briefly, following 60 minute BCAO, SPION-sODN antisense mmp9 (40 nmol/kg) or randomized sequence (40 nmol/kg) was infused ICV. MMP-9 expression and DWI/ADC were measured 7 hours following sODN infusion.

To demonstrate reduction of MMP-9 activity, gelatinase activity was measured using gelatin gel zymography. Extracts were prepared from the entire ipsilateral striatum of each animal and subjected to zymography as described (Gursoy-Ozdemir et al., J. Clin. Invest., 113:1447-1455, 2004). Four of five animals treated with SPION-Ran expressed gelatinase activity characteristic of activated MMP-9, whereas only one of seven animals treated with antisense SPION-mmp9 exhibited similar activity. No change was observed in the control protein actin. These data demonstrate that antisense sODN-mmp9 can reduce MMP-9 expression and/or activation following brain ischemia.

When antisense sODN (40 nmol/kg) was infused at two hours post BCAO, brain edema appeared in the striatum at 10 hours in 83% of animals that received SPION-Ran (N=6) and in 29% of animals that received antisense SPION-mmp9 (N=7). Although the percentages of animals that developed edema in each group were different, the edema volume in both groups at 24 hours did not differ from that of baseline animals.

Brain edema was reduced in animals that received antisense sODN-mmp9 (80 nmol/kg, striatal ADV=4.4, volume=4.8±0.9 mm³, N=1) without SPION at one hour of reperfusion. Edema volume in animals infused with sODN-Ran (6.22±0.84 mm³) did not differ significantly from baseline edema volume.

Quantitative analysis of ADC reduction was performed to determine metabolic disturbance in the striatum and cortex. A significant reversal of ADC drop was observed in the striatum, but not in the cortex, of animals treated with sODN-mmp9. Striatal volumes of metabolic disturbance (VMD) were compared in three groups of animals that received infusion of sODN-Ran or sODN-mmp9, or no infusion (No ICV control), after BCAO of 60 minutes. The VMD is defined by the region with an ADC value two SEM below the average ADC of normal animals. The striatal VMD in two control groups of animals, No ICV and sODN-Ran, were not significantly different (0.37±0.02 and 0.44±0.03 mm³, respectively). In the treated group that received BCAO and sODN-mmp9, a significant reduction by 30-40% to 0.27±0.04 mm³ was observed. These data indicate that BCAO-induced MMP-9 activities in mouse brain contributed to the development of metabolic disturbance and reduction of MMP-9 activity was sufficient to reduce tissue damage.

### Example 5

### Non-Invasive Delivery of Oligonucleotides

SPION-sODN once administered can be distributed through the lymphatic system. Experiments were performed to demonstrate delivery of SPION-mmp9 through intraperitoneal injection in animals that experience BBB disruption by BCAO.

SPION-mmp9, SPION-cfos, SPION-actin, SPION-Ran or an unlinked SPION and sODN mix (10 mg Fe per kg) were delivered i.p. to 10 animals after BCAO. We detected SPION retention in BCAO animals, but not in those that underwent a sham operation. When. SPION-mmp9 was delivered and MRI assessment performed fours days later to two animals, elevated R2* signal (indicative of probe retention) was observed in one of the two animals. Different SPION-sODNs showed different distributions: SPION-actin was observed throughout the entire brain, whereas SPION-cfos retention was noted in the cortex and striatum, and SPION-mmp9 retention was found in the striatum only. These distributions are similar to those observed with ICV infusion and BCAO treatment. Compared to baseline, no significant retention was observed when non-targeting SPION-Ran or unconjugated SPION was delivered i.p.

### Example 6

### MRI Detection of Brain Damage/Repair in Living Subjects

SPION-sODN once administered can be distributed through the lymphatic system. Experiments were performed to demonstrate delivery of SPION-mmp9 through intraperitoneal (i.p.) injection in animals that experience BBB disruption by BCAO.

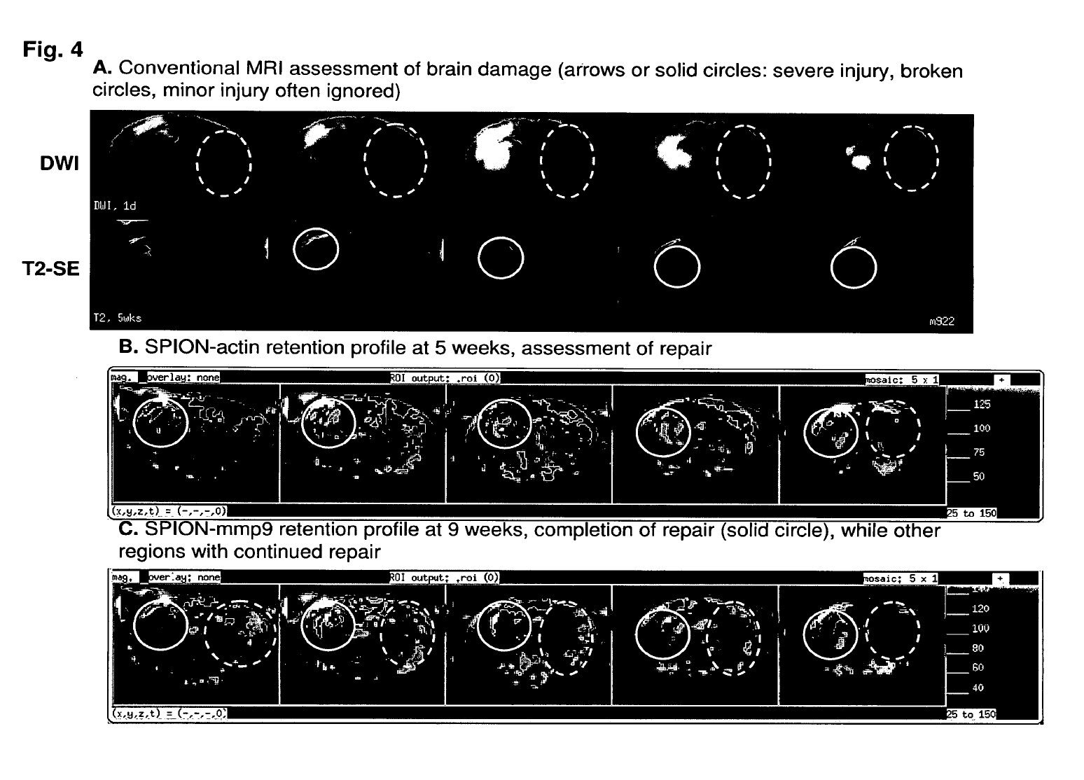

Six animals were subjected to cerebral ischemia by BCAO of 60 minutes. Shown in FIG. 4 are the results from one representative animal with a severe damage in the left hemisphere. DWI was acquired one day after reperfusion to detect abnormal water movement, which showed obvious hyperintensity (FIG. 4A, top row). T2 weighted images were acquired at five weeks to assess obvious physical damage in the brain. Severe ventriculmegaly and atrophy were found in the left hemisphere (FIG. 4A, bottom row). SPION-sODN (SPION-actin and SPION-mmp9) were applied serially to this animal at 5 and 9 weeks and SPION retention data were acquired the next day. Elevated SPION-actin uptake was observed throughout the brain, and specific focal retention around the enlarged ventricles (white circles, FIGS. 4B and C). Expression of actin in these cells also indicates stem cell activity from pericyte with multipotent cell types (Dore-Duffy et al. (2006) J. Cereb. Blood Flow Metab., 26:613-624). Four weeks later, SPION-mmp9 was administered and the retention profile of SPION-mmp9 was localized but reduced in sizes in the injured site, where cells expressing actin mRNA had been detected before (solid white circles, FIG. 4C). Because actin and MMP-9 are expressed in cells during angiogenesis (Costa et al. (1999) Am. J. Pathol., 155:1671-79; Raymond et al. (2004) J. Vasc. Surg., 40:1190-98), matched SPION retention in the brain detected by SPION-actin and SPION-mmp9 show sites of ongoing angiogenesis and brain repair in the right hemisphere which was subjected to a milder damage (dashed white circles). On the other hand, severe damage to the left hemisphere resulted in absence of SPION-mmp9 retention compared to SPION-actin, particularly in the cortical region where severe atrophy was obvious. These results suggest that necrotic cell death has occurred and is beyond repair in these regions. The presence of pericytes after SPION-actin detection were also found (Liu et al 2008, FASEB J., 22:1193-1203).

### Example 7

### Brain Damage in Living Mice Having Global Cerebral Ischemia

In this example, as well as the Examples 8-11 below, mice having GCI were further examined. The mice were generated in the manner described in Example 2 above. The MRI acquisition for DWI/ADC and R2* maps were performed in as described in Liu, et al. 2007 J Neurosci 27:713-722. Diffusion-weighted MR imaging was conducted on a total of 35 mice at various time points after reperfusion, from 1 hour and up to 6 days after BCAO. We measured (bilaterally) cortical and striatal VMD in each animal. MRI scans for the same animal were separated by at least seven hours and no scan was conducted on the same animal less than seven hours after a previous scan so as to avoid undue exposure to anesthesia during the recovery period. In animals that received FITC-sODN (see below), DWI was acquired and ADC changes were calculated. In animals that received SPION-sODN via the ICV route, however, we acquired DWI but did not measure ADC changes because the presence of SPION resulting from uptake causes signal reduction, which interferes with ADC calculation.

Protocols for in vivo MRI at 9.4T:(a) Iron assessment and R2* imaging: Total scan time for each animal was approximately 30 minutes. Animals were anesthetized in the manner describe above. The MRI protocols included: (1) RARE tripilot acquisition sequence for localization (2) Serial, 2D Gradient Echo Fast Imaging (GEFI) with TR/TE=500/3, 4, 6, 8, 10 ms, 117×117 μm2, 20 0.5 mm contiguous slices, flip angle=30, number of average (NA)=2.

(b) Gd-enhanced MRI: To detect BBB leakage, animals were scanned before the injection of Gd-DTPA (Magnevist; Shering, Berlin, Germany) using T1-weighted 3-D spin echo images (TR/TE=400/11 ms, 120×120×500 μm³, NA=2). Gd-DTPA was administered to the jugular vein (0.1 mM/kg) and imaged within 10 minutes. Areas of extensive enhanced T1 signals compared to the pre-Gd scans indicating leaky brain areas.

(c) Data processing for MR Image alignment, T2* (or R2*) maps calculation and ROI analysis: For voxel-wise and region-of-interest (ROI) comparison, images were automatically and manually aligned using nine degrees of freedoms (3 each): rotations, translations, and inflations. Fine-tuning of alignment was performed by visual comparison to the template images, focusing on obvious anatomical structure s, such as the corpus callosum and outlines of the ventricles. R2* maps were constructed from the aligned images (with incremental TEs). R2* (inverse of T2*) maps were calculated using pixel-wise linear fitting from the set of images with same TR and incremental TEs based on equation M=M0×exp(−TE/T2*). Elevated R2* (or reduced T2*) is, theoretically, caused by the presence of SPION in the tissue. ROI was outlined according to ‘The Mouse Brain in Stereotaxic Coordinates’ (Paxinos G. and Franklin K. B. J., 2001). We extracted the averaged R2* value within ROIs in each animal and calculate the group mean and standard error of the mean (SEM) were calculated in each group for statistical analysis.

Statistical analysis: Power calculation was performed using the average and standard error from the data obtained from the first set of animals; we calculated the number of animals in each group required to achieve 90% power for a p value of 0.02, according to an in-house software (La Morte W W, “Sample Size Calculation in Animal Research” Boston University Medical Center IACAUC Newsletter). Unless the power calculation called for a greater number of animals, each study was repeated with three animals in each treatment group. We computed the mean and standard error of the mean (SEM) from the averages in each group of animals, and determined the statistical significance of these values using at test (one-tailed, type II or equal variant) or contingency tests (Fisher's Exact test for p values, GraphPad Prism IV, GraphPad Software, Inc, San Diego, Calif.). A p value of less than 0.05 was statistically significant.

It was found that intracellular calcium and glutamate after GCI caused abnormal water retention which was detected by MR as hDWI and quantification using rADC in living brains. One week after GCI, neither metabolic disturbance nor vasogenic edema was detectable. However, BBB leakage and ventriculomegaly developed several-week later. To quantitatively compare the severity of metabolic disturbance after GCI in vivo, we measured the volume of metabolic disturbance (VMD) from MRI. To this end, we outlined the area of the brain with ADC values below the threshold of ADC which is two SEM below the average ADC from normal mice, or ADC at <2.5% (one tail) of the distribution curve of normal ADC (Liu et al. 2007. Mol Imaging 6:156-170). The summation of the product of the area below the threshold ADC and the thickness of the MRI slice in one mouse is the VMD.

Average VMDs at different time periods from at least four mice in each time point were obtained and charted to show a temporal VMD profile after 60 min BCAO in the cortex and striatum. It was found that VMD was the highest immediately after reperfusion among all time points, and was followed by a gradual drop in the VMD between 3-8 hours of reperfusion. VMD then rose gradually after 9 hours and reached a plateau between 9 and 48 hours in the striatum, followed by a second drop in volume beyond two days. The same peak was also observed in the cortex, but the plateau of cortical VMD was reached between 9 hours and 5 days. We also observed that brain regions with significant VMD after the 60-minute BCAO and reperfusion could appear both unilaterally (n=2, or 25%) and bilaterally (n=6, or 75%) in one series of experiments. Although Gd-MRI (Magnivist, 0.1 mmol/kg, intravenous injection) detected BBB leakage immediately after GCI, we found that all mice with significant striatal VMD at one day of reperfusion developed BBB leakage within the same hemisphere. Such BBB leakage in the striatum, but not those in the cortex, lasted approximately 12 weeks after GCI.

The VMD implies a significant GCI-induced brain damage within the boundary of severe rADC. Our further study suggested that reducing VMD by hypothermia (33±1° C.) during GCI also reduced MMP-9 expression and that hypothermia reduces neurological deficit in survivors who suffered heart attack/cardiac arrest.

### Example 8

### The Retention of SPION-mmp9 after GCI in Mice

We aimed to compare the expression of mmp-9 mRNA transcript in the cortex and the striatum in living brains using MRI. We conjugated sODN-mmp9 (and sODN-Ran as a control) to SPION via biotin and NeutrAvidin (NA) linkage. The SPION-mmp9 and SPION-Ran probes (Fe=40 μg or SPION=120 pmol per kg) were delivered by ICV infusion to the left lateral cerebral ventricle of the brain in two groups of mice after GCI; we chose ICV route of delivery to avoid variations due to BBB leakage. Group averaged R2* maps were computed based on MR images at 10 hr after reperfusion in the experimental and control groups to assess SPION probe retention. We observed localized elevation in R2* values, indicative of increased iron concentration, in the ischemic brains that received SPION-mmp9 than those received SPION-Ran.

Although the probe was infused into the left ventricle, we found no significant difference in the R2* values between the left and right hemispheres within individual animals in groups that received either SPION-mmp9 (between cortices, p=0.09; between striata, p=0.18), or SPION-Ran (p>0.25 in both cortex and striatum). Statistical analysis of regional SPION retention showed that retention of SPION-mmp9, compared to SPION-Ran, was significant elevated in both the striatum and cortex at 10 hours of reperfusion; still the elevated SPION-mmp9 retention was higher in the striatum than in the cortex. SPION-Ran retention was not significantly different from baseline measures in either SO or normal animals. The retention of SPION-mmp9 suggested that the expression of striatal mmp-9 mRNA was twofold greater in GCI animals than was cortical mmp-9 mRNA (less than two-fold increase).

### Example 9

### GCI Induced MMP-9 Activities and mRNA in the Regions of hDWI

In this example, MMP-9 expression was examined in the mouse brain after GCI. Because the cortex or striatum VMD reached a plateau at one day post GCI, DWI was acquired at that time GCI (n=5) and SO (n=2) in the manner described above. Immediately after MRI, postmortem brain samples were obtained for immunohistochemistry staining. Mouse MMP-9 antigen was stained using rabbit polyclonal antibodies against MMP-9 (ab38898, AbCam, Cambridge, Mass.) followed by FITC-anti-rabbit IgG as the secondary antibodies in the manner described in Gursoy-Ozdemir et al., 2004. J Clin Invest 113:1447-1455. Similarly, vascular endothelial cells were stained by cy3-griffonia simplicifolia lectin I and cell nuclei stained by Hoechst.

It was found that the mice exhibited MMP-9 immunoreactivity in the brain after GCI. Regions without hDWI in the same mice showed less or no expression of MMP-9 protein. No significant MMP-9 activities were observed in SO mice or in tissue from GCI mice without antibodies. The MMP-9 antigen (green) was located in cytoplasm and around the nuclei (purple) of cells non-endothelial cells as they were not stained with Cy3-griffonia simplicifolia lectin I. These results showed that MMP9 protein expression increased after GCI.

MMP-9 mRNA level was then determined in the same regions using a modified but sensitive ex vivo hybridization assay (Cui et al., 1999. J Neurosci 19:1335-1344.). FITC-labeled sODN-mmp9 or sODN-Ran was delivered using non-invasive route one hour after GCI as transient BBB leakage immediately following GCI allowed small molecules such as sODN-mmp9 or sODN-Ran to across the BBB. DWI was obtained the next day, followed by postmortem sample collection.

It was found that FITC-sODN-mmp9 was present in three of four GCI mice that received FITC-sODN-mmp9. Bilateral and unilateral hDWI/rADC from two of the mice were observed, providing a histological correlation between hDWI/rADC and the presence of FITC-sODN-mmp9/mRNA in both hemispheres (using the hippocampus as the reference point). Retention of FITC-ODN-mmp9 showed that leakage started from the vascular endothelial lumen, moved toward the parenchyma, and extended to cells at least 50 μm away from the vessels. In another mouse, Hoechst staining suggested loss of nuclear DNA one day post GCI, indicating cell damage in the dentate gyrus, where hDWI/rADC was observed. It was found that FITC-sODN-mmp9 was present in the cytoplasm of granule cells in the subgranular zone of the dentate gyms where hDWI/rADC was detected with MRI. However, no expression of MMP-9 mRNA was observed in the region where there was no abnormal DWI/ADC. Also, no FITC signal was found in any of the four mice having GCI and administered with FITC-sODN-Ran even though the mice did show hDWI with elevated MMP-9 activities. No MMP-9 mRNA was detected in the four mice that underwent SO.

The results indicated that BBB leakage allowed translocation of FITC-sODN to cerebral parenchyma and the above mmp9 brain probes had an intracellular specificity for target discriminations.

### Example 10

### Gene Targeting by Translation Blocking

Translation blocking was conducted by infusing sODN-mmp9 as a short inhibitory DNA (siDNA) at high dose (120 nmol/kg) via the ICV route after GCI. In this example, control groups included mice with sham operation or with GCI but no ICV.

Samples were collected from the tissue between the hippocampus and the olfactory bulbs—areas where hDWI was observed—for zymography of activated MMP-9. For each assay, 20 μg of protein from each mouse was used; all animals showed similar levels of actin protein in all samples. A positive control, C57black6 mouse brain sample after traumatic brain injury (TBI), was also included. This TBI sample had a higher level than that from GCI (both without ICV) and served as marker for mouse MMP-9. The results are summarized in Table 1 below.

TABLE 1
Statististical Analysis for animals with MMP-9 activities

| Groups | A | B | C | D | E |
|---|---|---|---|---|---|
| Treatments | GCI | Sham-operated + | Sham-operated | GCI | GCI |
| | No ICV | ICV saline | No ICV | sODN-Ran | sODN-mmp9 |
| Total animals tested (N) | 3 | 2 | 1 | 8 | 10 |
| Power Analysis | | | | | |
| Number of animals | 104 | | | 8 | 3 |
| between the groups^{!} | A vs D | | | D vs E | A vs E |
| Percent of animals that | 100% | 100% | 0 | 87.5% | 20% |
| show activated MMP-9 | | | | | |
| activities in zymograph | | | | | |
| P values (Chi-square | | | | | 0.0022** |
| test for zymograph) | | | | | (D vs E) |
| P values (Fisher exact | 0.035 | | | 0.6923 | 0.0076** |
| test for zymograph) | (A vs E) | | | (A vs D) | (D vs E) |
| VMD (mm³) | 37 ± 2 | 0 | 0 | 44 ± 3 | 27 ± 4* |

Data obtained from two separate experiments and a total of 24 mice at 10 hours reperfusion for the zymographs, with the exception of DWI/ADC.
^{!}Power analysis was performed as described therein.
*p = 0.04 (t test)

As shown in Table 1, no MMP-9 activation was observed in most animals that received GCI and sODN-mmp9 (Group E). In contrast, MMP-9 activities were detected in most animals that experienced GCI and received sODN-Ran (Group D). MMP-9 activities were present in seven of eight animals from the GCI and SPION-Ran group (87.5%), and in only two of 10 mice treated with GCI and sODN-mmp9 (20%). Fisher's exact test analysis indicated that the results of Group E differed significantly from those of the other groups (p<0.04) and that the results between Groups A and D were not statistically different (p>0.69). In addition, it was found that the effect of sODN-mmp9 on gene knockdown of MMP-9 activation was a short-term effect as no gene knockdown of MMP-9 activation was observed after 24 hours of delivery.

The above results indicate sODN-mmp-9 hybridized its target mRNA and knocked down MMP protein expression.

### Example 11

### Transient rADC Reversal in MRI after MMP-9 Knockdown

Assays were conducted to show that a reduction in MMP-9 activation diminishes the development of hDWI/rADC after GCI.

Briefly, the assays included using sODN-mmp9 at high dose as a siDNA after GCI in mice, and measuring the effect on hDWI/rADC. MR scans of the animals were acquired to measure hDWI/rADC at three time points after ICV infusion. To quantitatively measure the effect of sODN-mmp9, we included data from all mice and did not exclude potential outliers in statistical analysis. We observed a significant reversal of ADC drop in the striatum at one day in the group that received sODN-mmp9, compared to those received no ICV (n=11) or sODN-Ran (n=6). No change in cortical rADC was observed at this time point in all groups.

We then measured total VMD in mice using siDNA for gene knockdown of mmp-9 mRNA. We observed a significant reduction in VMD (30-40%) in the group that received sODN-mmp9, as compared to two different control groups: no ICV and sODN-Ran (Table 1). In repeated experiments using the same protocol (paired observations), five of six animals (83%) that received GCI and sODN-Ran exhibited normal hDWI at the same time points.

The above results show that sODN-mmp9 has multiple applications, such as MR imaging of in vivo target gene activities, ex vivo hybridization, and gene knockdown.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features. From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

1. A method of detecting a cellular matrix metalloprotease (MMP) nucleic acid in a tissue in vivo, the method comprising
obtaining a reporter conjugate comprising an MMP targeting nucleic acid linked to a reporter group, wherein the targeting nucleic acid hybridizes to a target MMP nucleic acid molecule corresponding to the cellular MMP nucleic acid to be imaged;
administering the reporter conjugate to the tissue in an amount sufficient to provide a detectable image;
allowing sufficient time to pass to allow a sufficient amount of unbound reporter conjugate to leave the tissue; and
imaging the tissue, wherein a detectable image of the reporter group in the tissue indicates the presence of the cellular MMP nucleic acid.

2. The method of claim 1, wherein the target MMP nucleic acid molecule comprises an MMP messenger RNA transcribed from a target MMP gene, and the targeting MMP nucleic acid comprises an antisense strand that hybridizes to a portion of the MMP messenger RNA, wherein the presence of the cellular nucleic acid indicates expression of the MMP target gene.

3. The method of claim 2, wherein the target MMP gene is matrix metalloprotease 2 (MMP-2) or matrix metalloprotease 9 (MMP-9).

4. The method of claim 1, wherein the tissue is brain tissue.

5. The method of claim 1, wherein the tissue is heart, lung, liver, pancreas, spinal cord, prostate, breast, gastrointestinal system, ovary, or kidney tissue.

6. The method of claim 1, wherein the reporter group is a superparamagnetic iron oxide particle whose maximum diameter is between 1 nm and 2000 nm.

7. The method of claim 1, wherein the tissue is in a human patient.

8. The method of claim 1, wherein the reporter conjugate is administered by intravenous injection.

9. The method of claim 1, wherein the reporter conjugate is administered via intra-cerebroventricular infusion.

10. A reporter conjugate for imaging a cellular nucleic acid comprising a single targeting matrix metalloprotease (MMP) nucleic acid linked to one or more paramagnetic iron oxide particles whose maximum diameter is between 1 nm and 1000 nm.

11. The reporter conjugate of claim 10, wherein the particle is a monocrystalline iron oxide nanoparticle (MION), ultra small superparamagnetic iron oxide particle (USPIO), or cross-linked iron oxide (CLIO) particle.

12. The reporter conjugate of claim 10, wherein the maximum diameter of the particle is between 10 nm and 100 nm.

13. The reporter conjugate of claim 10, further comprising cross-linked dextran surrounding the particle.

14. A method of treating a matrix metalloprotease (MMP)-mediated disorder or injury in a subject, the method comprising
obtaining a targeting MMP nucleic acid, wherein the targeting nucleic acid decreases expression or activity of a target MMP protein; and
administering the targeting MMP nucleic acid to the subject in an amount sufficient to decrease expression or activity of the target MMP protein, thereby treating the MMP-mediated disorder or injury.

15. The method of claim 14, wherein the MMP-mediated disorder or injury is stroke, head trauma, multiple sclerosis, bacterial meningitis, an HIV-associated neurological disease, or a cancer.

16.-20. (canceled)

21. The method of claim 7, wherein the human patient has an MMP-mediated disorder.

22. The method of claim 21, wherein the MMP-mediated disorder is stroke, head trauma, multiple sclerosis, bacterial meningitis, an HIV-associated neurological disease, or a cancer.

23.-25. (canceled)

26. The reporter conjugate of claim 10, wherein the reporter conjugate consists essentially of a single targeting matrix metalloprotease nucleic acid linked to one or more paramagnetic iron oxide particles.

27. The reporter conjugate of claim 10, wherein the nucleic acid is linked to the particles via a bridge agent that is covalently linked to the nucleic acid or the particles.

28. (canceled)
